(19) United States
(12) Patent Application Publication
(10) Pub. No.: US 2026/0306845 A1
(43) Pub. Date: Oct. 8, 2026

(54) IMPLANTABLE LEAD FOR OBSTRUCTIVE SLEEP APNEA

(71) Applicant: Medtronic, Inc., Minneapolis, MN (US)

(72) Inventors: Sean P. Skubitz, Forest Lake, MN (US); Adam J. Rivard, Blaine, MN (US); Jeffrey M. Novotny, North Branch, MN (US); Kelly N. Salb, Minneapolis, MN (US); Richard T. Stone, Roseville, MN (US); Thomas W. Hovind, Andover, MN (US); Robert R. Rowe, Monticello, MN (US); Jacob W. Silverberg, Blaine, MN (US)

(21) Appl. No.: 19/478,853

(22) PCT Filed: Apr. 10, 2024
(86) PCT No.: PCT/IB2024/053503

§ 371 Date: Oct. 27, 2025

Related U.S. Application Data
(60) Provisional application No. 63/498,235, filed on Apr. 25, 2023.

(51) Int. Cl. A61N 1/36; A61N 1/05

(52) U.S. Cl. CPC A61N 1/36078; A61N 1/0548; A61N 1/3611

(57) ABSTRACT

An implantable lead configured to be placed near a nerve of a patient, comprising: an elongated shaft comprising: a plurality of electrodes disposed on the elongated shaft; a plurality of flexible regions, each flexible region defining a concave cylindrical shape along the longitudinal axis and separating longitudinally adjacent electrodes of the plurality of electrodes, and wherein the concave cylindrical shape comprises: a distal portion defining a first diameter; a proximal portion defining a second diameter; and a concave portion connecting the distal portion and the proximal portion and defining a third diameter at a narrowest location along the concave portion; and a conductor coil disposed within the elongated shaft, and wherein each electrode of the plurality of electrodes is electrically connected to a respective conductive wire of the conductor coil.

This Application claims priority from U.S. Provisional Patent Application 63/498,235, filed 25 Apr. 2023, the entire content of which is incorporated herein by reference.

### TECHNICAL FIELD

This disclosure relates to medical device systems and, more particularly, to medical device systems for delivery of electrical stimulation therapy.

### BACKGROUND

Obstructive sleep apnea (OSA), which encompasses apnea and hypopnea, is a disorder in which breathing may be irregularly and repeatedly stopped and started during sleep, resulting in disrupted sleep and reduced blood oxygen levels. Muscles in a patient's throat intermittently relax thereby allowing soft tissues of the throat to obstruct the upper airway while sleeping and cause OSA. Airflow into the upper airway can be obstructed by the tongue or soft pallet moving to the back of the throat and covering the airway. Loss of air flow also causes unusual inter-thoracic pressure as a person tries to breathe with a blocked airway. Lack of adequate levels of oxygen during sleep can contribute to abnormal heart rhythms, heart attack, heart failure, high blood pressure, stroke, memory problems, and increased accidents during the day due to inadequate sleep. Additionally, loss of sleep occurs when a person is awakened during an apneic episode.

### SUMMARY

In general, devices, systems, and techniques of this disclosure generally relate to implantable leads for therapy for obstructive sleep apnea (OSA) but can be extended to address other patient symptoms and disorders. With OSA, a patient's tongue may relax during sleep and block the patient's airway. Some example techniques to address OSA include electrically stimulating one or both hypoglossal nerves and/or motor points in the tongue of the patient. In response to the electrical stimulation, the hypoglossal nerve(s) causes protrusor muscles (e.g., genioglossus and geniohyoid muscles) to contract and move the tongue forward, thereby opening the airway. In some examples, in response to stimulating at the motor points of the protrusor muscles (e.g., a location where an axon of the hypoglossal nerve terminates at a muscle fiber), the protrusor muscles may contract to move the tongue forward, thereby opening the airway.

To stimulate the hypoglossal nerve(s) and/or motor points, a medical device outputs electrical stimulation therapy via one or more electrodes on one or more implanted leads to cause the tongue to move forward. A medical professional can implant the one or more leads into the tongue of the patient using a needle. The one or more implanted leads each include one or more electrodes coupled to the medical device (e.g., an implantable or external medical device that delivers electrical stimulation via one or more electrodes on the lead).

During stimulation, the one or more implanted leads may repeatedly flex, e.g., in conjunction with the movement of the tongue. The cycles of flexure may increase stress and/or strain at particular locations on the implanted lead, e.g., at junctions between the one or more electrodes and a flexible material defining the remainder of each implanted lead. The high stress and/or strain may cause unintended separation of an electrode from the flexible material and/or reduce an lifespan of an implanted lead, thereby necessitating additional medical procedures to replace implanted leads and/or the medical device.

Issues with cycles of flexure may be prominent in leads implanted in the tongue due to normal activity (e.g., talking, eating, etc.). Accordingly, while the example implantable leads may be utilized for various treatment, and the example techniques should not be considered limited, the examples are described with respect to using the implantable leads for OSA treatment, where there is a relatively large curvature of the lead body or distal end of the lead during some of the flexure cycles of the implanted leads as compared to lead implant use condition of other patient therapies.

This disclosure describes example designs for implantable leads to increase the resistance of an implantable lead to wear and/or degradation from repeated cycles of flexure. In some examples, this disclosure describes designs for increasing the fixation between electrodes and flexible material(s) defining the body of the implantable lead, thereby increasing a number of cycles of stress and/or strain a junction between an electrode and the flexible material(s) may withstand. In some examples, this disclosure describes designs for increasing the flexure capabilities of electrodes, thereby reducing the stress and/or strain on the junction between the electrodes and the flexible material(s). In some examples, this disclosure describes designs for flexible material(s) between adjacent electrodes, thereby reducing the stress and/or strain on the junctions between each electrode and the flexible material(s). The example designs should not necessarily be considered as being exclusive. Accordingly, a lead may be configured in accordance with one or more of the example designs described in this disclosure.

In some examples, the disclosure describes an implantable lead configured to be placed near a nerve of a patient, comprising: an elongated shaft defining a longitudinal axis and an inner lumen, wherein the elongated shaft comprises: a plurality of electrodes disposed on the elongated shaft; a plurality of flexible regions, each flexible region defining a concave cylindrical shape along the longitudinal axis, wherein each flexible region separates longitudinally adjacent electrodes of the plurality of electrodes, and wherein the concave cylindrical shape comprises: a distal portion defining a first diameter; a proximal portion defining a second diameter; and a concave portion connecting the distal portion and the proximal portion and defining a third diameter at a narrowest location along the concave portion, wherein the third diameter is less than the first diameter or the second diameter; and a conductor coil disposed within the inner lumen and extending along the longitudinal axis, wherein the conductor coil comprises a plurality of conductive wires, and wherein each electrode of the plurality of electrodes is electrically connected to a respective wire of the plurality of conductive wires.

In some examples, the disclosure describes an implantable lead configured to be placed near a nerve of a patient, comprising: an elongated shaft defining a longitudinal axis and comprising: a plurality of electrodes; and a plurality of separate flexible regions arranged along the longitudinal axis, each flexible region extending along the longitudinal axis from a proximal end to a distal end and comprising: a first portion defining a first outer diameter; a second portion defining a second outer diameter; and a third portion connected to the first portion and the second portion and defining a third outer diameter, wherein the third outer diameter is less than the first outer diameter or second outer diameter.

In some examples, the disclosure describes a method of manufacturing an implantable lead configured to be placed near a nerve of a patient, comprising: forming an electrically conductive material into a plurality of electrodes, each electrode of the plurality of electrodes defining an elongated body configured to extend along a longitudinal axis of the lead; and forming an elongated shaft of the implantable lead along a longitudinal axis, the elongated shaft comprising: the plurality of electrodes; and a plurality of flexible regions connecting the plurality of electrodes, each flexible region of the plurality of flexible regions being configured to be disposed between longitudinally adjacent electrodes of the plurality of electrodes, and wherein each flexible region comprises: a distal portion defining a first diameter; a proximal portion defining a second diameter; and a medial portion connecting the distal portion and the proximal portion and defining a third diameter at a narrowest location along a longitudinal length of the flexible region, wherein each flexible region defines a concave cylindrical shape along the longitudinal length of the flexible region.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram of an implantable medical device (IMD) system for delivering obstructive sleep apnea (OSA) therapy.

FIG. 2 is a block diagram illustrating example configurations of implantable medical devices (IMDs) which may be utilized in the system of FIG. 1.

FIG. 3 is a block diagram illustrating an example configuration of an external programmer.

FIG. 4A is a conceptual diagram illustrating an example implantable lead of FIG. 1.

FIG. 4B is a cross-sectional diagram illustrating a cross-sectional view of the implantable lead of FIG. 4A along a longitudinal axis of the implantable lead of FIG. 4A.

FIG. 5A is a cross-sectional diagram illustrating a cross-sectional view of the implantable lead of FIG. 4A with an example electrode design, the cross-section being taken along the longitudinal axis of the implantable lead of FIG. 4A.

FIG. 5B is a cross-sectional diagram illustrating a cross-sectional view of the example electrode design of FIG. 5A.

FIG. 6A is a cross-sectional diagram illustrating a cross-sectional view of the implantable lead of FIG. 4A with another example electrode design, the cross-section being taken along the longitudinal axis of FIG. 4A.

FIG. 6B is a cross-sectional diagram illustrating a cross-sectional view of one example of the electrode design of FIG. 6A.

FIG. 6C is a cross-sectional diagram illustrating a cross-sectional view of another example of the electrode design of FIG. 6A.

FIG. 7A is a conceptual diagram illustrating another example implantable lead of FIG. 1.

FIG. 7B is a cross-sectional diagram illustrating a cross-sectional view of the implantable lead of FIG. 7A along a longitudinal axis of the implantable lead of FIG. 7A.

FIG. 8A is a cross-sectional diagram illustrating a cross-sectional view of an example electrode of the implantable lead of FIG. 4A, the cross-section being taken along a longitudinal axis of the implantable lead of FIG. 4A.

FIG. 8B is a cross-sectional diagram illustrating is a cross-sectional diagram illustrating a cross-sectional view of another example of the electrode of the implantable lead of FIG. 4A, the cross-section being taken along a longitudinal axis of the implantable lead of FIG. 4A.

FIG. 8C is a cross-sectional diagram illustrating a cross-sectional view of another example electrode of the implantable lead of FIG. 4A, the cross-section being taken along a longitudinal axis of the implantable lead of FIG. 4A.

FIG. 8D is a conceptual diagram illustrating flexure of the example electrode of FIG. 8A.

FIG. 8E is a conceptual diagram illustrating flexure of the example electrode of FIG. 8C.

FIG. 9A is a cross-sectional diagram illustrating another example cross-sectional view of the implantable lead of FIG. 4A along a longitudinal axis of the implantable lead of FIG. 4A.

FIG. 9B is a cross-sectional diagram illustrating a cross-sectional view of an example junction of the implantable lead of FIG. 9A, the cross-section being taken along a longitudinal axis of the implantable lead of FIG. 9A.

FIG. 9C is a cross-sectional diagram illustrating a cross-sectional view of another example electrode of the implantable lead of FIG. 4A, the cross-section being taken along a longitudinal axis of the implantable lead of FIG. 4A.

FIG. 9D is a cross-sectional diagram illustrating a cross-sectional view of another example electrode of FIG. 4A, the cross-section being taken along a longitudinal axis of the implantable lead of FIG. 4A.

FIG. 10 is a cross-sectional diagram illustrating a cross-sectional view of another example electrode design of the implantable lead of FIG. 4A, the cross-section being taken along a longitudinal axis of the implantable lead of FIG. 4A.

FIG. 11 is a conceptual diagram illustrating another example implantable lead of FIG. 1.

FIG. 12 is a flow diagram illustrating an example process for implanting an implantable lead in tissue of a patient.

FIG. 13 is a flow diagram illustrating an example process for manufacturing an example implantable lead.

### DETAILED DESCRIPTION

Medical devices, systems, and techniques for delivering electrical stimulation to the protrusor muscles of the tongue for the treatment of obstructive sleep apnea (OSA) are described in this disclosure. Electrical stimulation is delivered to cause the tongue of a patient to enter an advanced state, during sleep, to avoid or reduce upper airway obstruction. As used herein, the term, “advanced state” with regard to the tongue refers to a position that is moved forward and/or downward compared to a non-stimulated position or a relaxed position of the tongue. The advanced state is a state associated with contraction (e.g., via innervation from nerves in response to electrical stimulation) of protrusor muscles of the tongue (also sometimes referred to as “protruder” muscles of the tongue) including the genioglossus and geniohyoid muscles. An advanced state may be the opposite of a retracted and/or elevated position associated with the contraction of the retractor muscles (e.g., styloglossus and hyoglossus muscles) which retract and elevate the tongue. Electrical stimulation is delivered to cause the tongue to move (e.g., by depolarizing the nerve(s) that innervate the genioglossus and/or geniohyoid muscles) and maintain an advanced state. As discussed above, the advanced state may prevent collapse or blockage of, open, or widen the upper airway of a patient to at least partially maintain or increase airflow (e.g., promote unrestricted airflow or at least reduced restriction of airflow during breathing).

A surgeon implants one or more leads that each include one or electrodes into the tongue such that the electrodes are proximate to a hypoglossal nerve and/or motor points (e.g., one or more locations where axons of the hypoglossal nerve terminate at respective muscle fibers of the protrusor muscles). For example, there are two hypoglossal nerves in the tongue of the patient. In one example, one lead may be used to stimulate (e.g., by delivering electrical stimulation through one or more electrodes of the lead) one of the two hypoglossal nerves, one lead may be used to stimulate both hypoglossal nerves, or two leads may be used, where each lead stimulates a respective one of the hypoglossal nerves. Stimulation of either or both hypoglossal nerves of the tongue can cause contraction of the protrusor muscles to reduce the effect of or prevent OSA.

There are multiple sets of motor points for each of the protrusor muscles on the left side and the right side. Each motor point may innervate one or more muscle fibers of the protrusor muscle. In one example, one lead may be used to stimulate motor points, including hypoglossal nerve, for the protrusor muscles on one side of the tongue, one lead may be used to stimulate motor points, including hypoglossal nerve, for protrusor muscles on both sides of the tongue, or two leads may be used, where each lead stimulates a respective set of motor points for the protrusor muscles on each side. Stimulation of either or both sets of motor points of the tongue can cause contraction of the protrusor muscles to reduce the effect of, or prevent, OSA.

This disclosure describes examples of techniques related to design and/or implantation of the one or more leads in the tongue for treatment of OSA. Although the example techniques are described with respect to OSA, the example techniques should not be construed as limited to OSA. Rather, the example techniques described in this disclosure may be applicable to lead design and/or implantation for treatment of various conditions, including lead design and/or implantation for treatment of conditions where the lead is implanted in a location other than the tongue.

Open surgeries may be performed to implant the one or more leads in a tongue of a patient for treating OSA. However, such open surgeries require dissection of tissue to expose one or more hypoglossal nerves and/or motor points for placement of the one or more leads immediately adjacent to or around the hypoglossal nerves and/or motor points in the tongue of the patient, which is relatively invasive and time-consuming.

In other examples, medical professionals may implant the leads by using a needle to form a path through the tissue of the patient to the hypoglossal nerves of the patient. The one or more leads may then be navigated through the paths to areas adjacent to the hypoglossal nerves and deliver stimulation signals to the hypoglossal nerves. In such examples, the clinician first creates an initial path using a needle and removes the needle from the patient once the initial path has been created. The clinician then advances an introducer attached to a dilator, e.g., over a guidewire or other similar guiding device, to dilate the initial path to an appropriate diameter for the lead and to determine the appropriate orientation for the electrodes of the lead. In some examples, the clinician uses the introducer and the dilator to form the path in the tissue and deliver electrical signals to and/or sense electrical signals from the tissue of the patient to determine a proper position of the lead within the tissue, e.g., without requiring the use of a standalone needle. The medical professional may then remove to the dilator and introducer from the patient and advance an implantable lead sheath into the dilated path. The implantable lead sheath may include an electrically insulative material and may be configured to electrically insulate some portions of the lead while allowing other portions of the lead to deliver stimulation signals to the hypoglossal nerve, e.g., through the one or more electrodes. Finally, the medical professional may insert the lead into an inner lumen of the implantable lead sheath and advance the lead through the inner lumen of the implantable lead sheath to the hypoglossal nerves of the patient. The medical professional may then remove the lead sheath to complete the implantation process.

Unlike the examples that require dissecting tissue or examples that require a needle, introducer, guidewire, and other such components, some example techniques utilize a needle configured to percutaneously insert into skin and form a path for inserting a lead, such as without requiring the use of the guidewire or introducer. For instance, a surgeon may implant one or more leads adjacent to or around one or more hypoglossal nerves and/or motor points in the tongue of a patient without dissecting tissue to expose the hypoglossal nerves and/or motor points, which minimize access incision, shorten recovery time for the patient, and reduce risk for misplacement of the leads.

The electrical stimulation of the hypoglossal nerves and/or motor points in the tongue cause flexure of the tongue and, by extension, flexure of the one or more implantable leads. Flexure of the one or more implantable leads may lead to increase stress and/or strain along specific portions of each implantable lead, e.g., at junctions connecting electrodes to flexible material(s) defining the remainder of the implantable lead body. Repeated cycles of flexure may lead to wear and/or separation at the junctions along the implantable lead, thereby reducing the lifespan of the implantable lead possibly resulting in replacement of the implantable lead.

This disclosure describes example lead designs for an implantable lead configured to be implanted within a tongue of a patient. The example lead designs may include electrode designs and flexible material designs configured to reduce stress and/or strain at specific locations along the lead body (e.g., at the junctions between electrodes and flexible material(s)). The redistribution of the stress and/or strain experienced by the implantable lead may reduce the wear experienced by the implantable lead over time and increase an operational lifespan of the implantable lead.

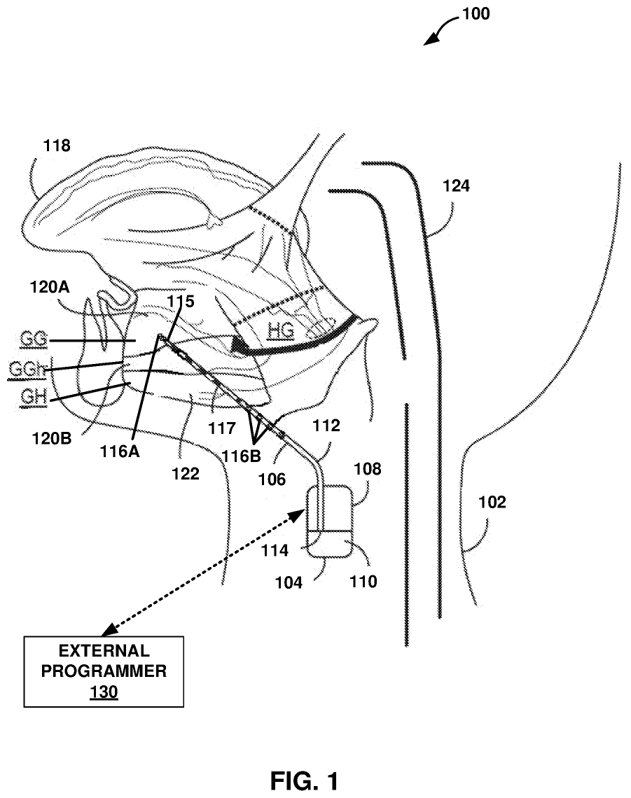

FIG. 1 is a conceptual diagram of a medical system for delivering OSA therapy. In system 100, implantable medical device (IMD) 104 and lead 106 are implanted in patient 102. IMD 104 includes housing 108 enclosing circuitry of IMD 104. In some examples, IMD 104 includes connector assembly 110, which is sealed to housing 108 and includes one or more connector bores for receiving a proximal end of at least one medical electrical lead 106 (also referred to as “implantable medical lead 106”, “lead 106”) used for delivering OSA therapy. Although one lead 106 is illustrated in FIG. 1, there may be one or more leads 106 to which IMD 104 is coupled.

Lead 106 may include a flexible, elongated lead body 112 (also referred to as “elongated member 112”) extending from lead proximal end 114 to lead distal end 115. As illustrated in FIG. 1, lead 106 includes one or more electrodes 117 that are carried along a lead distal portion adjacent lead distal end 115 and are configured for insertion within the protrusor muscles 120A, 120B, and 122 of tongue 118. As one example, the genioglossus muscle includes oblique compartment 120A and horizontal compartment 120B. In this disclosure, the genioglossus muscle is referred to as protrusor muscle 120. Protrusor muscle 122 is an example of the geniohyoid muscle. Lead body 112 includes one or more electrodes 117 and one or more fixation elements 116A-B (Collectively referred to as “one or more fixation elements 116”). In some examples, protrusions, indentations, creases, or other texturing may be disposed on the outer surface of lead body 112 and/or between electrodes 117. The texturing may increase friction between the tissue of patient 102 and lead body 112 and may prevent dislodgement of lead 106 within the tissue.

Proximal end 114 of lead 106 includes one or more electrical contacts to connect to connector assembly 110. Lead 106 also includes conductors such as coils or wires that connect respective electrodes 117 to respective electrical contacts at proximal end 114 of lead 106.

Once the clinician determines that lead 106 is properly placed within protrusor muscles 120 and/or 122, the clinician may secure lead 106 within protrusor muscles 120 and/or 122 via one or more fixation elements 116. For instance, the one or more fixation elements 116 may be in an undeployed state, due a sheath covering the one or more fixation elements 116, while the clinician positions lead 106. The clinician may then remove the sheath to deploy the one or more fixation elements 116.

Tongue 118 includes a distal end (e.g., tip of tongue 118), and electrodes 117 may be implanted proximate to a root of tongue 118. The surgeon may implant one or more leads 106 such that one or more electrodes 117 are implanted proximate to the root of tongue 118, as illustrated in FIG. 1. For example, the location for stimulation for the genioglossus muscle 120 may be approximately 30 mm (e.g., 25 mm to 35 mm) from the symphysis of the jaw (e.g., where the genioglossus and hypoglossal muscles insert). The location for stimulation for the geniohyoid muscle 122 may be approximately 40 mm (e.g., 35 mm to 45 mm) from the symphysis. For both the genioglossus muscle 120 and the geniohyoid muscle 122, the location for stimulation may be approximately 11 mm (e.g., 7 mm to 15 mm) lateral to the midline on both the right and left sides of tongue 118 for stimulating respective hypoglossal nerves. In some examples, rather than or in addition to stimulating hypoglossal nerves, the examples described in this disclosure may be configured for stimulating the motor points.

Stimulating the motor points may result in indirect activation of the hypoglossal nerve but may generally be stimulating at a different location than direct stimulation to the hypoglossal nerve. As a result, in some examples, simulation of one or more motor points may result in more precise activation of muscle fibers than may be possible with stimulation of the hypoglossal nerve itself. However, in some examples, it may be possible to stimulate at the hypoglossal nerve, or stimulate both the motor points and the hypoglossal nerve.

One or more electrodes 117 of lead 106 may be ring electrodes, segmented electrodes, partial ring electrodes, or any suitable electrode configuration. Ring electrodes extend 360 degrees around the circumference of lead body 112 of lead 106. Segmented and partial ring electrodes each extend along an arc less than 360 degrees (e.g., 90-120 degrees) around the outer circumference of lead body 112 of lead 106. In this manner, multiple segmented electrodes may be disposed around the perimeter of lead 106 at the same axial position of the lead. In some examples, segmented electrodes may be useful for targeting different fibers of the same or different nerves at respective circumferential positions with respect to the lead to generate different physiological effects (e.g., therapeutic effects), permitting stimulation to be oriented directionally. In some examples, lead 106 may be, at least in part, paddle-shaped (e.g., a “paddle” lead), and may include an array of electrodes arranged as contacts or pads on a common surface, which may or may not be substantially flat and planar.

As described above, in some examples, electrodes 117 and/or distal electrode 119 of lead 106 are disposed within the musculature of tongue 118. Accordingly, one or more electrodes 117 of lead 106 may be “intramuscular electrodes.” Intramuscular electrodes may be different than other electrodes that are placed on or along a nerve trunk or branch, such as a cuff electrode, used to directly stimulate the nerve trunk or branch. The example techniques described in this disclosure are not limited to intramuscular electrodes and may be extendable to electrodes placed closer to a nerve trunk or branch of the hypoglossal nerve(s). Also, in some examples, one or more electrodes 117 of lead 106 may be implanted in connective tissue or other soft tissue proximate to the hypoglossal nerve.

Electrical stimulation therapy generated by IMD 104 and delivered via one or more electrodes 117 and/or the distal electrode 119 may activate protrusor muscles 120 and 122 to move tongue 118 forward, for instance, to promote a reduction in obstruction or narrowing of the upper airway 124 during sleep. As used herein, the term “activated” with regard to the electrical stimulation of protrusor muscles 120 and 122 refers to electrical stimulation that causes depolarization or an action potential of the cells of the nerve (e.g., hypoglossal nerve(s)) or stimulation at the neuro-muscular junction between the nerve and the protrusor muscles (e.g., at the motor points) innervating protrusor muscles 120 and 122 and motor points and subsequent depolarization and mechanical contraction of the protrusor muscle cells of protrusor muscles 120 and 122. In some examples, protrusor muscles 120 and 122 may be activated directly by the electrical stimulation therapy.

Protrusor muscles 120 and/or 122, on a first side of tongue 118 (e.g., the left or right side of tongue 118), may be activated by a medial branch or more distal end of a first hypoglossal nerve, and the protrusor muscles, on a second side of tongue 118 (e.g., the other of the left or right side of tongue 118), may be activated by a medial branch or more distal end of a second hypoglossal nerve. The medial branch of a hypoglossal nerve may also be referred to as the XIIth cranial nerve. The hyoglossus and styloglossus muscles (not shown in FIG. 1), which cause retraction and elevation of tongue 118, are activated by a lateral branch of the hypoglossal nerve.

One or more electrodes 117 and/or distal electrode 119 may be used to deliver bilateral or unilateral stimulation to protrusor muscles 120 and 122 via the medial branch of the hypoglossal nerve or branches of the hypoglossal nerve (e.g., such as at the motor point where a terminal branch of the hypoglossal nerve interfaces with respective muscle fibers of protrusor muscles 120 and/or 122). The examples of the location of delivery of stimulation is provided as examples, and should not be considered limiting.

For example, one or more electrodes 117 and/or distal electrode 119 may be coupled to output circuitry of IMD 104 to enable delivery of electrical stimulation pulses in a manner that selectively activates the right and left protrusor muscles (e.g., in a periodic, cyclical, or alternating pattern) to avoid muscle fatigue while maintaining upper airway patency. Additionally, or alternatively, IMD 104 may deliver electrical stimulation to selectively activate protrusor muscles 120 and/or 122 or portions of protrusor muscles 120 and/or 122 during unilateral stimulation of the left or right protrusor muscles.

In some examples, one lead 106 may be implanted such that one or more of electrodes 117 and/or distal electrode 119 may deliver electrical stimulation to stimulate the left hypoglossal nerve or motor points of protrusor muscles on the left side of tongue, and therefore cause the left protrusor muscles to activate. In such examples, the electrical stimulation from one or more electrodes 117 and/or distal electrode 119 may not be of sufficient amplitude to stimulate the right hypoglossal nerve or motor points of protrusor muscles on the right side of tongue and cause the right protrusor muscles to activate. In some examples, one lead 106 may be implanted such that one or more of electrodes 117 and/or distal electrode 119 delivers electrical stimulation to stimulate the right hypoglossal nerve or motor points of protrusor muscles on the right side of tongue, and therefore cause the right protrusor muscles to activate. In such examples, the electrical stimulation from one or more electrodes 117 and/or distal electrode 119 may not be of sufficient amplitude to stimulate the left hypoglossal nerve or motor points of protrusor muscles on the left side of tongue and cause the left protrusor muscles to activate. Accordingly, in some examples, two leads like lead 106 may be implanted to stimulate each of the left and right hypoglossal nerves and/or motor points of respective protrusor muscles on the left and right side of tongue 118.

In some examples, one lead 106 may be implanted substantially in the middle (e.g., center) of tongue 118. In such examples, one or more electrodes 117 and/or distal electrode 119 may deliver electrical stimulation to both hypoglossal nerves or motor points of both muscles on both sides of tongue 118, causing both hypoglossal nerves or motor points to activate respective left and right protrusor muscles. It may be possible to utilize current steering and field shaping techniques such that one or more electrodes 117 and/or distal electrode 119 deliver first electrical stimulation that stimulates the left hypoglossal nerve or motor points of protrusor muscles on the left side of tongue 118 with little to no stimulation of the right hypoglossal nerve or motor points of protrusor muscles on the right side of tongue 118, and then one or more electrodes 117 and/or distal electrode 119 deliver second electrical stimulation that stimulates the right hypoglossal nerve or motor points of protrusor muscles on the right side of tongue with little to no stimulation of the left hypoglossal nerve or motor points of protrusor muscles on the left side of tongue. In examples where two leads like lead 106 are utilized, each lead may alternate delivery of stimulation to respective hypoglossal nerves or motor points. In this way, IMD 104 may stimulate one hypoglossal nerve or one set of motor points and then the other hypoglossal nerve or another set of motor points, which can reduce muscle fatigue.

For instance, continuous stimulation may cause protrusor muscles to be continuously in an advanced state. This continuous contraction may cause protrusor muscles 120 and/or 122 to fatigue. In such cases, due to fatigue, the stimulation may not cause protrusor muscles 120 and/or 122 to maintain an advanced state (or higher intensity of the electrical stimulation may be needed to cause protrusor muscles 120 and/or 122 to remain in the advanced state). By stimulating one set of protrusor muscles (e.g., left or right), a second set (e.g., other of left or right) of protrusor muscles can be at rest. Stimulation may then alternate to stimulate the protrusor muscles that were at rest and thereby maintain protrusion of tongue 118 while permitting the protrusor muscles 120 and/or 122 that were previously activated to rest. Hence, by cycling between alternate stimulation of the left and right protrusor muscles, tongue 118 can remain in the advanced state, while one of the first or second set of protrusor muscles is at rest.

In some examples, one lead 106 may be implanted laterally or diagonally across tongue 118 such that some of electrodes 117 and/or distal electrode 119 on lead 106 can be used to stimulate the left hypoglossal nerve and/or motor points of the protrusor muscles on the left side of tongue 118 and some of electrodes 117 and/or distal electrode 119 on the same lead 106 can be used to stimulate the right hypoglossal nerve and/or motor points of the protrusor muscles on the right side of tongue 118. In such examples, IMD 104 may selectively deliver electrical stimulation to a first hypoglossal nerve and/or first motor points of the protrusor muscles on a first side of tongue 118 via a first set of one or more electrodes 117 and/or distal electrode 119, and then deliver electrical stimulation to a second hypoglossal nerve and/or second set of motor points of the protrusor muscles on a second side of tongue 118 via a second set of one or more electrodes 117 and/or distal electrode 119. This may be another way in which to reduce muscle fatigue.

Tongue 118 may transition between the advanced state (e.g., in response to the electrical stimulation by IMD 104) and an un-advanced (e.g., relaxed) state. The transition between the advanced and un-advanced states may cause lead 106 to flex and/or bend, e.g., in response to movement of the tissue of tongue 118 surrounding lead 106. In addition, normal movement of tongue 118 (e.g., due to eating, talking, etc.) may cause lead 106 to flex and/or bend in response to movement of the tissue of tongue 118. Each transition from the advanced state to the un-advanced state or vice versa may be referred to herein as a “flexure cycle”. Repeated flexure cycles over time may cause wear along lead 106 (e.g., at junctions between electrodes 117 and a flexible material defining lead body 112), e.g., due to the repeated application and concentration of stress and/or strain at specific locations along lead 106 (e.g., at the junctions).

The example designs for lead 106 described in this disclosure may include leads 106 having electrodes 117 and/or lead body 112 being configured to redistribute the stress and/or strain experienced by lead 106 during flexure cycles. The example lead designs may also reduce a magnitude of the stress and/or strain at the specific locations along lead 106 (e.g., at the junctions). The redistribution and/or reduction of the stress and/or strain applied to lead 106 may reduce an amount of wear on lead 106 due to the flexure cycles and/or increase a lifespan of lead 106.

Lead proximal end 114 includes a connector (not shown in FIG. 1) that may be coupled to connector assembly 110 of IMD 104 to provide electrical connection between circuitry enclosed by the housing 108 of IMD 104. Lead body 112 encloses electrical conductors extending from each of one or more electrodes 117 and/or distal electrode 119 to the proximal connector at proximal end 114 to provide electrical connection between output circuitry of IMD 104 and the electrodes 117.

There may be various ways in which lead 106 is implanted in patient 102. A clinician may insert the needle through the lower part of the jaw and in tongue 118 starting from the back of tongue 118. The clinician may insert the needle until a distal tip of the needle reaches a point at or adjacent to the root of tongue 118, angling the needle to extend proximate to the hypoglossal nerve (e.g., left or right hypoglossal nerve). In some examples, the needle may include one or more electrically conductive areas (e.g., one or more electrodes) at the distal end, and the clinician may cause the one or more electrically conductive areas of the needle to output electrical stimulation (e.g., in the form of controlled current pulses or controlled voltage pulses), which in turn causes a physiological response such as activation of protrusor muscles 120 and/or 122 and advancement of tongue 118. In some examples the one or more electrodes may be disposed on an outer surface of the needle. The clinician may adjust the location of the needle based on the physiological response to determine a location in tongue 118 that provides effective treatment.

In some examples, once the needle is implanted within tongue 118, the clinician may retract needle from within tongue 118, advance an introducer into tongue 118 via a path formed by the needle, and advance lead 106 through the introducer lumen of the introducer. The clinician may determine lead 106 is at a desired position within tongue 118, secure lead 106 within tongue 118 via one or more fixation elements 116, and retract the introducer from within tongue 118 once lead 106 is secured within tongue 118.

IMD 104 may output stimulation signals through electrodes 117 to stimulate the hypoglossal nerve and/or one or more motor points of the protrusor muscle within tongue 118. If further refinement is needed to determine the lead placement for lead 106, the clinician may adjust the location of needle and/or lead 106 within patient 102 in response to one or more electrical signals detected by electrodes 117 and/or electrodes on the needle. During implantation and testing of lead 106, IMD 104 may not yet be implanted within body of patient 102. After completing implantation and testing of lead 106, the clinician may implant IMD 104 within patient 102 (e.g., in the neck of patient 102, in the torso of patient 102, or the like) to complete the implantation of system 100. In some examples, lead 106 may be connected to another computing device and/or system (e.g., an external programming device) and the other computing device and/or system may output the stimulation signals for purposes of delivering the lead placement for lead 106.

As an example, some other techniques of implanting lead 106 include using a needle to percutaneously insert into the skin. A clinician places a guidewire through the lumen of needle, then removes the needle. The guidewire remains in place in the tissue in original location as the needle inside patient 102. The clinician places an introducer sheath, possibly with a dilator, over the guidewire. The clinician removes the guidewire, and places lead 106 into the introducer sheath. The clinician engages one or more fixation elements 116A-B (collectively referred to as “one or more fixation elements 116”) with the tissue of patient 102. Once the one or more fixation elements 116 are engaged, the clinician then removes the introducer sheath leaving lead 106 in place. The one or more fixation elements 116 may include, but are not limited to, a distal fixation element 116A disposed at lead distal end 115 or one or more fixation elements 116B (e.g., fixation tines) disposed along lead body 112 (e.g., between longitudinally adjacent electrodes 117, proximal to electrodes 117).

In any of the manners described above, the surgeon may implant one lead 106. In examples where two or more leads are implanted, the surgeon may perform steps similar to those described above.

The above describes some example techniques for lead placement, and the examples described in this disclosure should not be considered limited to such examples of lead placement. Moreover, in some examples, the surgeon may use imaging techniques, such as fluoroscopy, during implantation to verify proper placement of lead 106, the needle, and/or the introducer.

FIG. 1 illustrates the location of IMD 104 as being within or proximate to the neck of patient 102. However, IMD 104 may be implanted in various other locations. As one example, the surgeon may implant IMD 104 in the left or right pectoral region. For instance, the surgeon may plan on implanting IMD 104 in the left pectoral region unless another medical device is already implanted in the left pectoral region. If another medical device is already implanted in the left pectoral region, the surgeon may then implant IMD 104 in the right pectoral region. There may include other locations where the surgeon may implant IMD 104, such as the back of patient 102. The example techniques are not limited to any particular implant location of IMD 104.

In accordance with one or more examples described in this disclosure, system 100 is an implant system for utilizing lead 106 in tongue 118 for treatment of OSA. In some examples, system 100 may be configured such that substantial dissection is not required to expose one or more hypoglossal nerves and/or one or more motor points of the protrusor muscle within tongue 118 for placement of the lead. In some examples, system 100 may be configured such that a surgeon may implant the needle and lead 106 within patient 102 using a relatively smaller number of devices (e.g., without the use of an introducer sheath, guide members (e.g., a guidewire), a dilator, and the like).

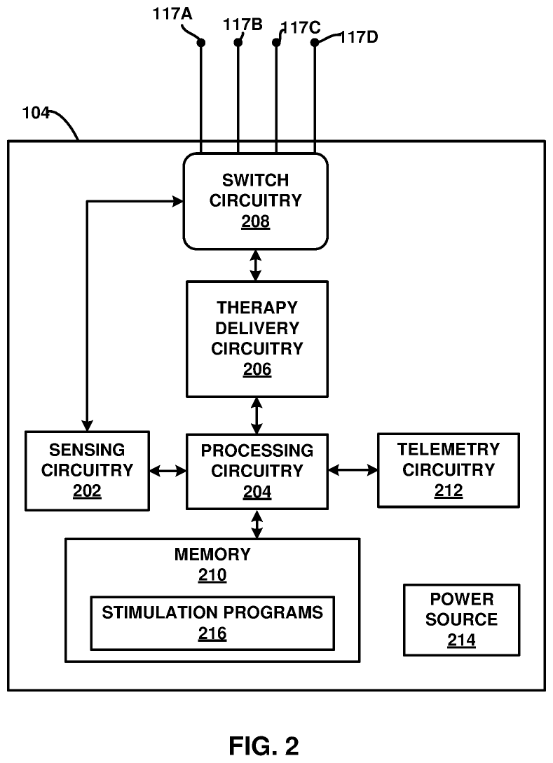

FIG. 2 is block diagram illustrating example configurations of implantable medical devices (IMDs) which may be utilized in the system of FIG. 1. As shown in FIG. 2, IMD 104 includes sensing circuitry 202, processing circuitry 204, therapy delivery circuitry 206, switch circuitry 208, memory 210, telemetry circuitry 212, and power source 214. IMD 104 may include a greater or fewer number of components. For example, in some examples, such as examples in which IMD 104 deliver the electrical stimulation in an open-loop manner, IMD 104 may not include sensing circuitry 202.

Switch circuitry 208 may be configured to, in response to instructions from processing circuitry 204, switch the coupling of one or more of electrodes 117A-D (also referred to as “electrodes 117”) between sensing circuitry 202 and therapy delivery circuitry 206. In examples where sensing circuitry 202 is not used, switch circuitry 308 may not be needed. However, even in examples where sensing circuitry 202 is not used, IMD 104 may include switch circuitry 208, e.g., to disconnect electrodes 117 from therapy delivery circuitry 206.

In some examples, therapy delivery circuitry 206 may include a plurality of regulated current sources or sinks, with each current source or sink coupled to one of electrodes 117. In such examples, therapy delivery circuitry 206 may control each current source or sink and switching between electrodes 117 may not be necessary for therapy delivery since each one of electrodes 117 is individually controllable.

Although not shown in FIG. 2, in some examples, IMD 104 may include one or more sensors configured to sense posture or position of patient 102. For example, IMD 104 may include accelerometer to determine if patient 102 is lying down. Another example of the one or more sensors is a motion sensor, and movement sensed by the motion sensor may indicate if patient 102 is having restless sleep, which may be indicative of the onset of OSA. Additional examples of the sensors include acoustical sensors or a microphone for detecting vibrations in upper airway 124. Vibrations in upper airway 124 may be indicative of the onset of OSA. In some examples, processing circuitry 204 may control delivery of therapy based on information received from the one or more sensors, such as delivery of therapy after sensing an onset of OSA.

In some examples, electrodes 117 may be configured to sense electromyogram (EMG) signals. Sensing circuitry 202 may be switchably coupled to electrodes 117 via switch circuitry 208 to be used as EMG sensing electrodes with electrodes 117 are not being used for stimulation. EMG signals may be used by processing circuitry 204 to detect sleep state and/or low tonal state of protrusor muscles 120 and/or 122 for use in delivering electrical stimulation. In some examples, rather than using electrodes 117 or in addition to using electrodes 117, there may be other electrodes or sensors used to sense EMG signals.

In general, IMD 104 may comprise any suitable arrangement of hardware, alone or in combination with software and/or firmware, to perform the techniques attributed to IMD 104 and processing circuitry 204, therapy delivery circuitry 206, and telemetry circuitry 212 of IMD 104. In various examples, IMD 104 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components.

The various units of IMD 104 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, one or more of the units may be distinct circuit blocks (fixed-function or programmable), and in some examples, one or more of the units may be integrated circuits.

IMD 104 may include arithmetic logic units (ALUs), elementary function units (EFUs), digital circuits, analog circuits, and/or programmable cores, formed from programmable circuits. In examples where the operations of IMD 104 are performed using software executed by the programmable circuits, memory 210 may store the instructions (e.g., object code) of the software that processing circuitry 204 receives and executes, or another memory within IMD 104 (not shown) may store such instructions.

IMD 104 also, in various examples, may include a memory 310, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Moreover, although sensing circuitry 202, processing circuitry 204, therapy delivery circuitry 206, switch circuitry 208, and telemetry circuitry 212 are described as separate circuitry, in some examples, sensing circuitry 202, processing circuitry 204, therapy delivery circuitry 206, switch circuitry 208, and telemetry circuitry 212 are functionally integrated. In some examples, sensing circuitry 202, processing circuitry 204, therapy delivery circuitry 206, switch circuitry 208, and telemetry circuitry 212 correspond to individual hardware units, such as ASICs, DSPs, FPGAs, or other hardware units.

Memory 210 stores stimulation programs 216 (also called “therapy programs 216”) that specify stimulation parameter values for the electrical stimulation provided by IMD 104. Memory 210 may also store instructions for execution by processing circuitry 204, in addition to stimulation programs 216. Information related to sensed parameters of patient 102 (e.g., from sensing circuitry 202 or the one or more sensors of IMD 104) may be recorded for long-term storage and retrieval by a user, and/or used by processing circuitry 204 for adjustment of stimulation parameters (e.g., amplitude, pulse width, and pulse rate). In some examples, memory 210 includes separate memories for storing instructions, electrical signal information, and stimulation programs 216. In some examples, processing circuitry 304 may select new stimulation parameters for a stimulation program 216 or new stimulation program from stimulation programs 216 to use in the delivery of the electrical stimulation based on patient input and/or monitored physiological states after termination of the electrical stimulation.

Generally, therapy delivery circuitry 206 generates and delivers electrical stimulation under the control of processing circuitry 204. In some examples, processing circuitry 204 controls therapy delivery circuitry 206 by accessing memory 210 to selectively access and load at least one of stimulation programs 216 to therapy delivery circuitry 206. For example, in operation, processing circuitry 204 may access memory 210 to load one of stimulation programs 216 to therapy delivery circuitry 206.

By way of example, processing circuitry 204 may access memory 210 to load one of stimulation programs 216 to control therapy delivery circuitry 206 for delivering the electrical stimulation to patient 102. A clinician or patient 102 may select a particular one of stimulation programs 216 from a list using a programming device, such as a patient programmer or a clinician programmer. Processing circuitry 204 may receive the selection via telemetry circuitry 212. Therapy delivery circuitry 206 delivers the electrical stimulation to patient 102 according to the selected program for an extended period of time, such as minutes or hours while patient 102 is asleep (e.g., as determined from the one or more sensors and/or sensing circuitry 202). For example, processing circuitry 204 may control switch circuitry 208 to couple electrodes 117 to therapy delivery circuitry 206.

Therapy delivery circuitry 206 delivers electrical stimulation according to stimulation parameters. In some examples, therapy delivery circuitry 206 delivers electrical stimulation in the form of electrical pulses. In such examples, relevant stimulation parameters may include a voltage or current pulse amplitude, a pulse rate, a pulse width, a duty cycle, and/or the combination of electrodes 117 that therapy delivery circuitry 206 uses to deliver the stimulation signal. In some examples, therapy delivery circuitry 206 delivers electrical stimulation in the form of continuous waveforms. In such examples, relevant stimulation parameters may include a voltage or current amplitude, a frequency, a shape of the stimulation signal, a duty cycle of the stimulation signal, or the combination of electrodes 117 therapy delivery circuitry 206 uses to deliver the stimulation signal.

In some examples, the stimulation parameters for the stimulation programs 216 may be selected to cause protrusor muscles 120 and/or 122 to an advanced state (e.g., to open-up airway 124). An example range of stimulation parameters for the electrical stimulation that are likely to be effective in treating OSA (e.g., upon application to the hypoglossal nerves to cause protrusor muscles 120, 122 to protrude or upon application to motor points), are as follows:
a. Frequency or pulse rate: between about 20 Hz and about 50 Hz, and possibly lower such as 2 Hz and 4 Hz. In some examples, the minimum target frequency is used which can achieve muscle tetany (e.g., constant contraction) and provide the required force to open the airway.
b. Current Amplitude: between about 0.1 milliamps (mA) and about 20 mA, and more generally from 0.5 mA to 3 mA, and approximately 1.5 mA.
c. Pulse Width: between about 100 microseconds (us) and about 500 μs. In some examples, a pulse width of 150 μs might be used for reduced power consumption. In some particular examples, the pulse width is approximately 240 μs. In some cases, shorter pulse widths may be used in conjunction with higher current or voltage amplitudes.

Processing circuitry 204 may select stimulation programs 216 for alternating delivery of electrical stimulation between stimulating the left protrusor muscles 120 and/or 122 and the right protrusor muscles 120 and/or 122 on a time basis, such as in examples where two leads 106 are implanted. In some examples, there may be some overlap in the delivery of electrical stimulation such that for some of amount of time both left and right protrusor muscles 120 and/or 122 are being stimulated. In some examples, there may be a pause in alternating stimulation (e.g., stimulate left protrusor muscles, a time period with no stimulation, then stimulate right protrusor muscles, and so forth). Processing circuitry 204 may also select stimulation programs 216 that select between different combinations of electrodes 117 for stimulating, such as to stimulate different locations of the hypoglossal nerve(s), which may help with fatigue as well as provide more granular control of how much to protrude tongue 118.

In the example of FIG. 2, therapy delivery circuitry 206 drives electrodes 117 of lead 106. Specifically, therapy delivery circuitry 206 delivers electrical stimulation (e.g., regulated current or voltage pulses at pulse rates and pulse widths described above) to tissue of patient 102 via selected electrodes 117A-117D carried by lead 106. A proximal end of lead 106 extends from the housing of IMD 104 and a distal end of lead 106 extends to a target therapy site, e.g., through inner lumen of the needle. Target therapy sites may include one or both hypoglossal nerves and/or motor points. Therapy delivery circuitry 206 may deliver electrical stimulation with electrodes on more than one lead and each of the leads may carry one or more electrodes, such as when patient 102 is implanted with two leads 106 in tongue 118 for stimulating both hypoglossal nerves simultaneously or bilaterally (e.g., one after the other) or for stimulating multiple motor points simultaneously. The leads may be configured as an axial lead with ring electrodes or segmented electrodes and/or paddle leads with electrode pads arranged in a two-dimensional array. The electrodes may operate in a bipolar or multi-polar configuration with other electrodes, or may operate in a unipolar configuration referenced to an electrode carried by the device housing or “can” of IMD 104.

In some examples, processing circuitry 204 may control therapy delivery circuitry 206 to deliver or terminate the electrical stimulation based on patient input received via telemetry circuitry 212. Telemetry circuitry 212 includes any suitable hardware, firmware, software, or any combination thereof for communicating with another device, such as an external programmer. Under the control of processing circuitry 204, telemetry circuitry 212 may receive downlink telemetry (e.g., patient input) from and send uplink telemetry (e.g., an alert) to a programmer with the aid of an antenna, which may be internal and/or external. Processing circuitry 204 may provide the data to be uplinked to the programmer and the control signals for telemetry circuitry 212 and receive data from telemetry circuitry 212.

Generally, processing circuitry 204 controls telemetry circuitry 212 to exchange information with a medical device programmer and/or another device external to IMD 104. Processing circuitry 204 may transmit operational information and receive stimulation programs or stimulation parameter adjustments via telemetry circuitry 212. Also, in some examples, IMD 104 may communicate with other implanted devices, such as stimulators, control devices, or sensors, via telemetry circuitry 212.

Power source 214 delivers operating power to the components of IMD 104. Power source 214 may include a battery and a power generation circuit to produce the operating power. In some examples, the battery may be rechargeable to allow extended operation. Recharging may be accomplished through proximal inductive interaction between an external charger and an inductive charging coil within IMD 104. In other examples, an external inductive power supply may transcutaneously power IMD 104 whenever electrical stimulation is to occur.

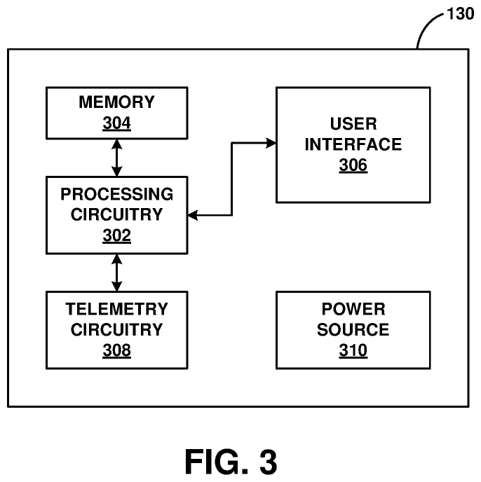

FIG. 3 is a block diagram illustrating an example configuration of an external programmer 130. While programmer 130 may generally be described as a hand-held computing device, programmer 130 may be a notebook computer, a cell phone, or a workstation, for example. As illustrated in FIG. 3, external programmer 130 may include processing circuitry 302, memory 304, user interface 306, telemetry circuitry 308, and power source 310.

In general, programmer 130 comprises any suitable arrangement of hardware, alone or in combination with software and/or firmware, to perform the techniques attributed to programmer 130, and processing circuitry 302, user interface 306, and telemetry circuitry 308 of programmer 130. Examples of processing circuitry 302 may include one or more processors, such as one or more microprocessors, DSPs, ASICs, FPGAs, or any other equivalent integrated or discrete logic circuitry, as well as any combinations of such components. Examples of memory 304 may include RAM, ROM, PROM, EPROM, EEPROM, flash memory, a hard disk, a CD-ROM, comprising executable instructions for causing the one or more processors to perform the actions attributed to them. Moreover, although processing circuitry 302 and telemetry circuitry 308 are described as separate circuitry, in some examples, processing circuitry 302 and telemetry circuitry 308 are functionally integrated. In some examples, processing circuitry 302 and telemetry circuitry 308 correspond to individual hardware units, such as ASICs, DSPs, FPGAs, or other hardware units.

In some examples, memory 304 may further include program information (e.g., stimulation programs) defining the electrical stimulation, similar to those stored in memory 210 of IMD 104. The stimulation programs stored in memory 304 may be downloaded into memory 210 of IMD 104.

User interface 306 may include a button or keypad, lights, a speaker for voice commands, a display, such as a liquid crystal (LCD), light-emitting diode (LED), or cathode ray tube (CRT). In some examples the display may be a touch screen. As discussed in this disclosure, processing circuitry 302 may present and receive information relating to electrical stimulation and resulting therapeutic effects via user interface 306. For example, processing circuitry 302 may receive patient input via user interface 306. The input may be, for example, in the form of pressing a button on a keypad or selecting an icon from a touch screen.

Processing circuitry 302 may also present information to the patient in the form of alerts related to delivery of the electrical stimulation to patient 102 or a caregiver via user interface 306. Although not shown, programmer 130 may additionally or alternatively include a data or network interface to another computing device, to facilitate communication with the other device, and presentation of information relating to the electrical stimulation and therapeutic effects after termination of the electrical stimulation via the other device

Telemetry circuitry 308 supports wireless communication between IMD 104 and programmer 130 under the control of processing circuitry 302. Telemetry circuitry 308 may also be configured to communicate with another computing device via wireless communication techniques, or direct communication through a wired connection. In some examples, telemetry circuitry 308 may be substantially similar to telemetry circuitry 212 of IMD 104 described above, providing wireless communication via an RF or proximal inductive medium. In some examples, telemetry circuitry 308 may include an antenna, which may take on a variety of forms, such as an internal or external antenna.

Examples of local wireless communication techniques that may be employed to facilitate communication between programmer 130 and another computing device include RF communication according to the 802.11 or Bluetooth specification sets, infrared communication (e.g., according to the IrDA standard), or other standard or proprietary telemetry protocols. In this manner, other external devices may be capable of communicating with programmer 130 without needing to establish a secure wireless connection.

Power source 310 delivers operating power to the components of programmer 130. Power source 310 may include a battery and a power generation circuit to produce the operating power. In some examples, the battery may be rechargeable to allow extended operation.

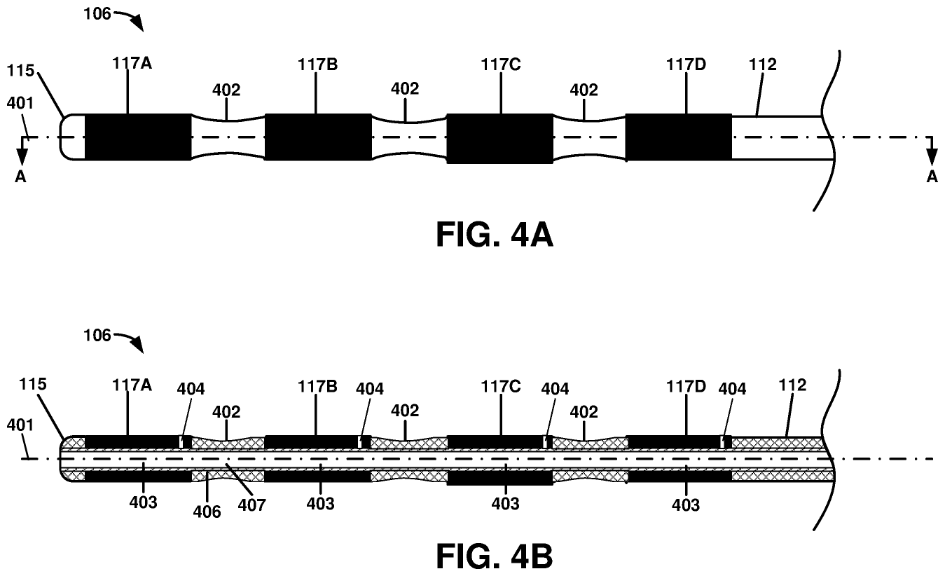

FIG. 4A is a conceptual diagram illustrating an example implantable lead 106 of FIG. 1. FIG. 4B is a cross-sectional diagram illustrating a cross-sectional view of implantable lead 106 of FIG. 4A along a longitudinal axis 401 of implantable lead 106. The cross-section of FIG. 4B is taken along line A-A, which may extend along longitudinal axis 401. As illustrated in FIGS. 4A and 4B, lead 106 includes lead body 112 extending along longitudinal axis 401 of lead 106 to lead distal end 115. A plurality of electrodes 117 are disposed on lead body 112 and along longitudinal axis 401. Lead body 112 includes a plurality of flexible portions 402 disposed along longitudinal axis 401. Each flexible portion 402 may be connected to two electrodes 117. While example lead 106 of FIGS. 4A and 4B includes four electrodes 117 (i.e., electrodes 117 A-D), other example leads may include one, two, three, or five or more electrodes 117. The other example leads may also include a corresponding number of flexible portions 402 depending on the number of electrodes 117 disposed on the example lead.

Lead body 112 and/or one or more flexible portions 402 may include one or more flexible material(s). The flexible material(s) may be configured to flex, e.g., along a plane orthogonal to longitudinal axis 401, in response to a force applied on a portion of lead body 112 and/or on one or more of electrodes 117. The force may be applied by tissue on tongue 118 and may be in response to movement of tongue 118 (e.g., by patient 102, in response to electrical stimulation). The flexible material(s) may include one or more biocompatible polymers.

As illustrated in FIG. 4B, the flexible material(s) may be disposed within an inner lumen 403 defined by electrodes 117 and connect longitudinally separated flexible portions 402. Each of electrodes 117 may define an outer surface and an inner surface. The inner surface of each of electrodes 117 may define inner lumen 403. For example, each of electrodes 117 may define an elongated annulus along longitudinal axis 401. An outer surface of the elongated annulus may define the outer surface of electrode 117 and an inner surface of the elongated annulus (e.g., a surface of the elongated annulus radially inwards of the outer surface) may define the inner surface of electrode 117 and the outer perimeter of inner lumen 403. In some examples, the flexible material(s) may connect flexible portions 402 to a portion of lead body 112 proximal to electrodes 117. The flexible material(s) may be disposed around an electrical conductor 406 disposed within lead body 112 and extending along longitudinal axis 401.

The flexible material(s) may include a plurality of materials having different properties. For example, lead body 112 and/or flexible portions 402 may include a first flexible material having a first durometer hardness and a second flexible material having a second durometer hardness. The durometers of the flexible material(s) may be about 50 Shore A to about 75 Shore D hardness (e.g., ±10-15%). In some examples, the first flexible material may define a first layer around conductor 406 and the second flexible material may define a second layer disposed radially outwards of the first layer. In some examples, the flexible material(s) may form one layer or three or more layers around conductor 406. In such examples, the first flexible material may have a higher durometer than the second flexible material, such that the second layer is more flexible than the first layer e.g., to facilitate flexibility and resilience of lead 106. In some examples, flexible portion 402 may be formed from a first flexible material and the portion of lead body 112 proximal to electrodes 117 may be formed from a second flexible material. In such examples, the second flexible material may have a higher durometer than the first flexible material, e.g., to increase flexibility of a distal portion of lead 106 containing electrodes 117. Each of the flexible material(s) may include a biocompatible polymer.

In some examples, the first layer is only disposed within inner lumen 403 of electrodes 117. In such examples, portions of flexible portion 402 within inner lumen 403 of electrodes 117 includes the first flexible material and the second flexible material and portions of the flexible portion 402 outside of inner lumen 403 of electrodes 117 includes the second flexible material and does not include the first flexible material. Restricting the first flexible material to be within inner lumen 402 of electrode 117 may increase flexibility of lead 106 and reduce the stress and/or strain on the connections between flexible portions 402 and electrodes 117.

Longitudinally adjacent electrodes 117 may be affixed to a flexible portion 402 disposed between the two electrodes 117. Flexible portion 402 may provide flexibility to the distal portion of lead body 112, e.g., to allow for flexure or bending of lead 106 in response to movement of tongue 118 and muscles in the throat. Each flexible portion 402 may be connected to a first electrode of electrodes 117 (e.g., electrode 117A) at a distal end and to a second electrode of electrodes 117 (e.g., electrode 117B) at a proximal end. The ends of electrodes 117 may be connected to ends of flexible portions 402 at a plurality of junctions.

Flexible portions 402 may define an outer diameter less than equal to the outer diameter of electrodes 117, where electrodes 117 are ring or segment electrodes, e.g., to reduce an overall profile of lead 106. The longitudinal length of each of flexible portions 402 may vary, e.g., based on a desired implantation location in tongue 118 and/or placement of electrodes 117 in tongue 118. As illustrated in FIG. 4A, each of flexible portions 402 may define a groove along longitudinal axis 401, e.g., that that the outer diameter at a longitudinal center of each respective flexible portion 402 is less than the outer diameters of one or more electrodes 117 longitudinally adjacent to the respective flexible portion 402. In some examples, a center of the groove may define a minimum outer diameter of each flexible portion 402. In some examples, each flexible portion 402 may define a plurality of grooves along the longitudinal length of each respective flexible portion 402.

Each of electrodes 117 may define an elongated body extending along longitudinal axis 401. The elongated body may be entirely an electrically conductive material (e.g., an electrically conductive metallic alloy). The electrically conductive metallic alloy may include electrically conductive biocompatible metallic alloys including, but is not limited to, Titanium alloys, Tantalum alloys, and/or Tin alloys. In some examples, an electrically conductive material may be disposed over at least a portion of an outer surface of the elongated body.

Each of electrodes 117 may be electrically connected to IMD 104 via conductor 406. For example, each of electrodes 117 may be electrically coupled to conductor 406 via one or more conductive wires (not pictured) secured to electrode 117 (e.g., to an outer conductive surface of electrode 117, or to an inner conductive surface of electrode 117). The conductive wire may be permanently affixed to electrode 117, e.g., via welding, crimping, or the like. The conductive wire may be disposed within or affixed to electrode 117 within a corresponding opening 404. For example, the conductive wire may be welded or crimped to an inner surface defining opening 404. Opening 404 may be disposed towards one or more of a distal end or a proximal end of electrode 117 and/or towards a longitudinal center of electrode 117.

Conductor 406 may extend along longitudinal axis 401 from lead proximal end 114 to lead distal end 115. Conductor 406 may include a plurality of conductive wires defining a conductive coil. In some examples, conductor 406 may include an elongated tube extending along longitudinal axis 401 and include a plurality of conductive wires disposed on and wrapping around an outer perimeter of the elongated tube. Each of the plurality of conductive wires disposed on the outer perimeter may define a helix or coil. Each conductive wire may be coupled to one of electrodes 117 and may be directly connected to or indirectly connected (e.g., via an intermediary conductive wire) to the corresponding electrode 117. For example, a conductive wire defining conductor 406 may extend radially away from conductor 406 and directly connect to one of electrodes 117 via physical contact between the conductive wire and the corresponding electrode 117. In another example, the conductive wire defining conductor 406 may be in physical contact with an intermediary conductive wire, and wherein the intermediary conductive wire physically contacts the conductive wire at one end and the corresponding electrode 117 at another end to form an indirect connection between the conductive wire of conductor 406 and the corresponding electrode 117. In some examples, as illustrated in FIG. 4B, conductor 406 may define an inner lumen 407 extending along longitudinal axis 401 and being configured to retain one or more components, e.g., a needle, a guide element (e.g., a guidewire), or the like. Inner lumen 407 of conductor 406 may be disposed in the middle of conductor 406 and lead 106.

Conductor 406 may be configured to flex along a plane orthogonal to longitudinal axis 401, e.g., in response to movement of tongue 118 and muscles in the throat. Conductor 406 may have a similar flexibility to the flexible material(s) defining flexible portions 402 and/or lead body 112.

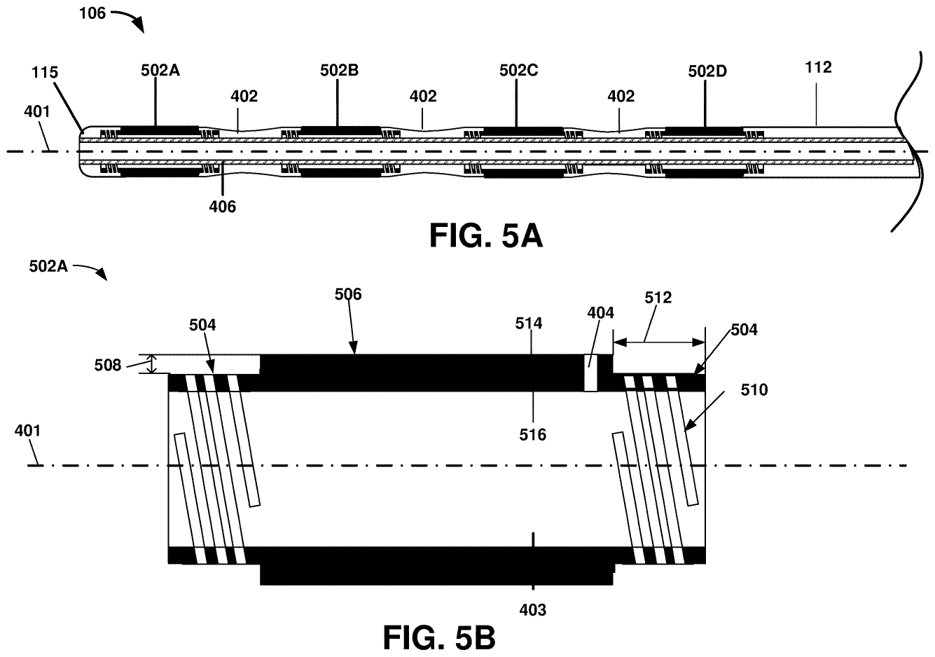

FIG. 5A is a cross-sectional diagram illustrating a cross-sectional view of the implantable lead 106 of FIG. 4A along longitudinal axis 401 of FIG. 4A. FIG. 5A illustrates lead 106 having example electrodes 502A-D (collectively referred to as “electrodes 502”) disposed along a longitudinal length of lead 106. FIG. 5B is a cross-sectional diagram illustrating a cross-sectional view of the example electrode 502 of FIG. 5A, the cross-section being taken along longitudinal axis 401 of FIG. 4A. As illustrated in FIGS. 5A and 5B, each of electrodes 502 may be connected to flexible portions 402. Each of electrodes 502 may define an elongated body 506 defining junctions 504 disposed at the ends of elongated body 506 (e.g., at distal and proximal ends of each of electrodes 502).

Junctions 504 may define a reduced diameter region of elongated body 506 (i.e., define a smaller outer diameter than elongated body 506). The thickness of elongated body 506 at junction 504 may differ from a maximum thickness of elongated body 506 (e.g., at a longitudinal center of elongated body 506) by a distance 508. Additionally, junction 504 may define a longitudinal length 512. Each of electrodes 502 having junctions 504 may have the same dimensions or having different dimensions. One or more of electrodes 502 may define junctions 504 having different dimensions than another of electrodes 502. For example, one or more of junctions 504 may define a different outer diameter. The change in outer diameter may be stepwise, as illustrated in FIG. 5B, or continuous (e.g., slopped).

In conjunction, distance 508 and longitudinal length 512 may define a volume encapsulating each junction 504. Each volume is configured to retain at least a portion of flexible portion 402 without increasing the overall outer diameter of lead 106. The volume encapsulating junction 504 increases a surface contact area between each electrode 502 and a connected flexible portion 402. The increased surface contact area may increase the strength of the bond between electrode 502 and flexible portion 402, thereby increasing the resistance of the bond to repeated cycles of stress and/or strain exerted upon the bond.

Each junction 504 may also include one or more openings 510 extending around the perimeter of junction 504. For each of electrodes 502, openings 510 may be disposed on a single junction 504, on both junctions 504, or on none of junctions 504. Each of electrodes 502 may define a different number of junctions 504, a different number of openings 510, and/or different arrangements of openings 510. A portion of flexible portion 402 may be disposed within openings 510, thereby further increasing the bond strength between each of electrodes 502 and adjacent flexible portions 402. In some examples, openings 510 may be configured to contract and expand in response to movement of tongue 118, thereby causing at least a part of each of electrodes 502 (e.g., junctions 504 of electrodes 502) to flex about a plane orthogonal to longitudinal axis 401. The flexure of at least a part of each of electrodes 502 may reduce stress and/or strain concentration at the bonds connecting electrodes 502 with flexible portions 402 and decrease wear of the bond.

One or more openings 510 may define a spiral or coil extending along longitudinal axis 401 and around the outer perimeter of junction 504. The spiral or coil may define a constant or variable pitch. In some examples, as illustrated in FIG. 5B, a single opening 510 defines the spiral or coil. In some examples, two or more openings 510 may define a partially interrupted spiral or coil. In some examples, one or more openings 510 may extend along a plane orthogonal to longitudinal axis 401. Each opening 510 may extend from an outer surface 514 of elongated body 506 to inner surface 516 of elongated body 506. In other examples, each opening 510 may extend partially from outer surface 514 towards inner surface 516 or vice versa.

Outer surface 514 of elongated body 506 may include an electrically conductive material. Each of electrodes 502 may be configured to deliver electrical signals to adjacent tissue of patient 102 via at least a portion of outer surface 514 of elongated body 506. Outer surface 514 may be electrically connected to conductor 406, e.g., via a conductive wire electrically coupled to outer surface 514. In some examples, the conductive wire may be electrically coupled to outer surface 514 via opening 404 extending from inner surface 516 at least partially towards outer surface 514. As illustrated in FIG. 5B, opening 404 may be located on a portion of elongated body 506 outside of junction 404, e.g., to allow for access (e.g., by a conductive wire) to outer surface 514.

Inner surface 516 may define inner lumen 403 of elongated body 506 extending along longitudinal axis 401. In some examples, as illustrated in FIG. 5A, inner lumen 403 may be configured to retain conductor 406 and portions of flexible portions 402 and/or flexible material(s) connecting longitudinally adjacent flexible portions 402.

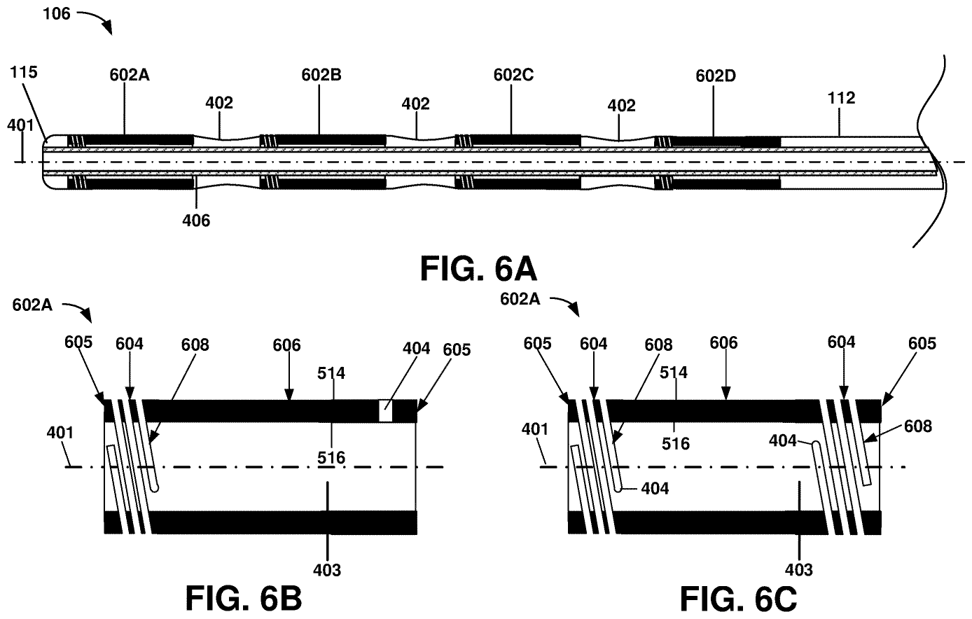

FIG. 6A is a cross-sectional diagram illustrating a cross-sectional view of the implantable lead 106 of FIG. 4B with example electrodes 602A-D, the cross-section being take along line A-A of FIG. 4A. FIG. 6B is a cross-sectional diagram illustrating a cross-sectional view of electrode 602 of FIG. 6A. FIG. 6C is a cross-sectional diagram illustrating a cross-sectional view of another example of electrode 602 of FIG. 6A.

Each of electrodes 602 may include an elongated body 606 defining one or more junctions 604. Each junction 604 may include one or more openings 608 configured to form a bond with flexible portion 402. For example, each opening 608 may retain a portion of flexible portion 402, thereby increasing a surface area between and increasing the strength of the bond between flexible portion 402 and junction 604. Junctions 604 may be configured to flex in response to movement of tongue 118 (e.g., similar to junctions 504 of electrode 502). Each of electrodes 602 may include one junction 604 disposed at one end (e.g., at a distal end) of elongated body 606, as illustrated in FIG. 6B, or two junctions 604 disposed at both ends of elongated body 606, as illustrated in FIG. 6C.

As compared to elongated bodies 506 of electrodes 502 of FIGS. 5A and 5B, elongated bodies 606 of electrodes 602 may define a constant outer diameter (i.e., junctions 604 do not define reduced-diameter portions of elongated bodies 606). The constant outer diameter may reduce a thickness of flexible portions 402 and/or increase flexibility of flexible portion 402.

An electrically conductive outer surface (e.g., outer surface 514) of each of electrodes 602 may be electrically coupled to conductor 406 via a conductive wire disposed within opening 404. Opening 404 may be disposed on elongated body 606 (e.g., around one end of elongated body 606, around a longitudinal center of elongated body 606). In some examples, as illustrated in FIG. 6B, opening 404 may be disposed around an end of elongated body 606 that does not define junction 604. In some examples, opening 608 may define one or more openings 404. For example, as illustrated in FIG. 6C, an end of each of openings 608 defines a corresponding opening 404 connecting inner surface 516 of elongated body 606 to outer surface 514 of elongated body 606. Opening 404 may have a same or different diameter and/or width than one or more of openings 608. Placement of one or more openings 404 at ends of openings 608 may allow for openings 608 to form spirals or coils around the outer perimeter of junctions 604 without necessitating interruption of the spirals or coils to form openings 404 in elongated body 606. Placement of openings 404 relatively further away from ends 605 of elongated body 606 may also reduce stress and/or strain applied to conductive wire affixed within openings 404, thereby reducing wear on the connections between elongated body 604 and conductor 406.

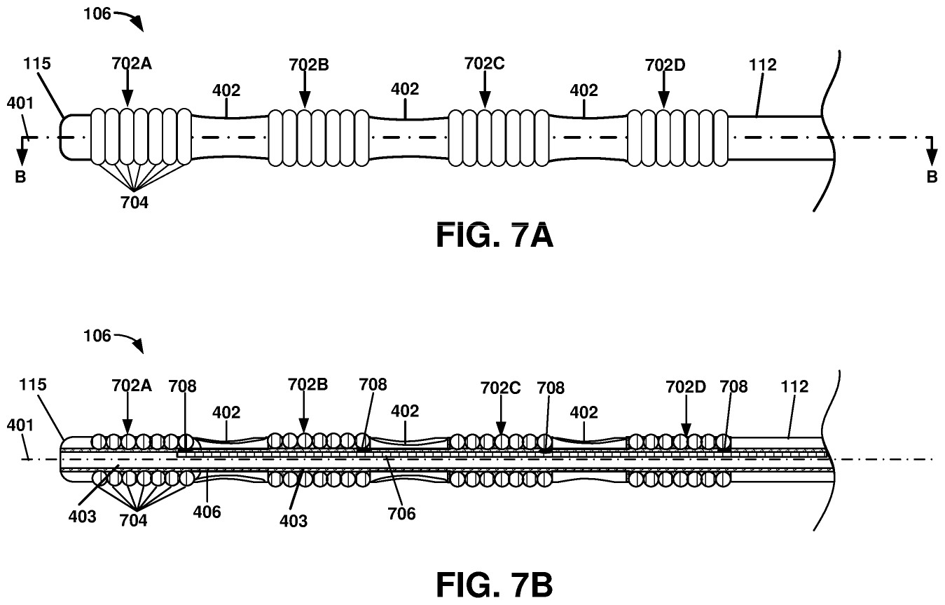

FIG. 7A is a conceptual diagram illustrating another example implantable lead 106 of FIG. 1. FIG. 7B is a cross-sectional diagram illustrating a cross-sectional view of the implantable lead 106 of FIG. 7A along longitudinal axis 401 of the implantable lead of FIG. 7A. As illustrated in FIG. 7A, the cross-section may be taken along line B-B, which may extend along longitudinal axis 401. As illustrated in FIGS. 7A and 7B, lead 106 may include a plurality of electrodes 702A-D disposed along the longitudinal length of lead body 112 of lead 106, longitudinally adjacent electrodes 702 being separated by a flexible portion 402, as previously discussed herein. Each of electrodes 702 may be formed by a conductive wire 704 shaped into a cylindrical shape and disposed on lead body 112. Each of electrodes 702 may define lumen 403 of lead body 112 which may retain conductor 406.

Forming electrodes 702 via conductive wire 704 may provide various benefits such as increased flexibility relative to other examples (e.g., having a solid elongated body). The increased flexibility may reduce the stress and/or strain exerted on connections between electrodes 702 and connected flexible portions 402, thereby reducing wear on the connections. Conductive wire 704 may be formed from electrically conductive materials including, but are not limited to, platinum-iridium, a nickel-cobalt alloy (e.g., MP35N, Ag/MP35N), stainless steel, or a titanium alloy (e.g., Ti-15Mo).

While the examples describe that electrodes 702 formed via conductive wire 704 may provide increased flexibility relative to other examples, the example techniques described in this disclosure should not be considered limiting. That is, it may be possible for electrodes 702 to have flexibility, but less flexibility than elongated electrodes. The flexibility of the electrodes 702 and elongated electrodes may be set by various factors, and can be controlled as part of the design.

Conductor 406 may include a plurality of conductive wires 706. Conductive wires 706 may define a spiral or coil extending along longitudinal axis 401. For each of electrodes 702, conductive wire 704 may be electrically coupled to one or more corresponding conductive wires 706 of conductor 406. In some examples, each conductive wire 704 may be a separate wire from the corresponding conductive wire 706 of conductor 406 and may be electrically coupled to the corresponding conductive wire 706 via a corresponding intermediary conductive wire 708. Intermediate conductive wire 708 may be affixed to conductive wire 704 at one end (e.g., via welding, crimping) and affixed to conductive wire 706 at the other end. In some examples, each conductive wire 704 and corresponding conductive wire 706 may be a single wire. In such examples, a distal portion of each conductive wire 706 may extend radially away from conductor 406 and wrap around lead body 112 to form a corresponding electrode 702. Forming electrodes 702 from the distal portions of conductive wires 706 may eliminate a connection between electrode 702 and conductor 406, thereby increasing the resistance of lead 106 to stress and/or strain. Electrodes 702 on lead 106 may include any combination of connections between conductive wires 704 and conductive wires 706.

In some examples, an implantable lead 106 may be configured to be placed near a hypoglossal nerve of a patient, comprising: an lead body 112 defining a longitudinal axis 401; and a plurality of electrodes 702 disposed on a distal portion of the lead body 112, each electrode 702 of the plurality of electrodes 702 comprising a conductive wire 704 defining a coil extending along the longitudinal axis 401 and between two longitudinally adjacent portions 402 of the lead body 112.

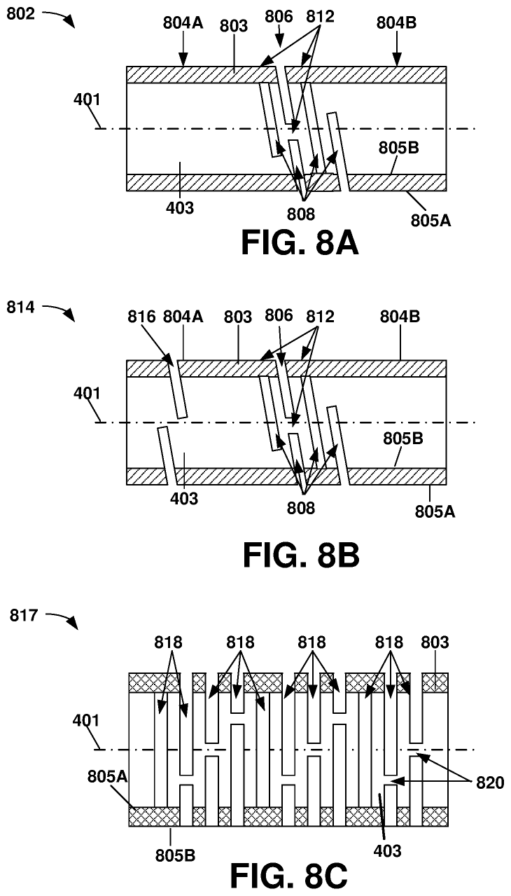

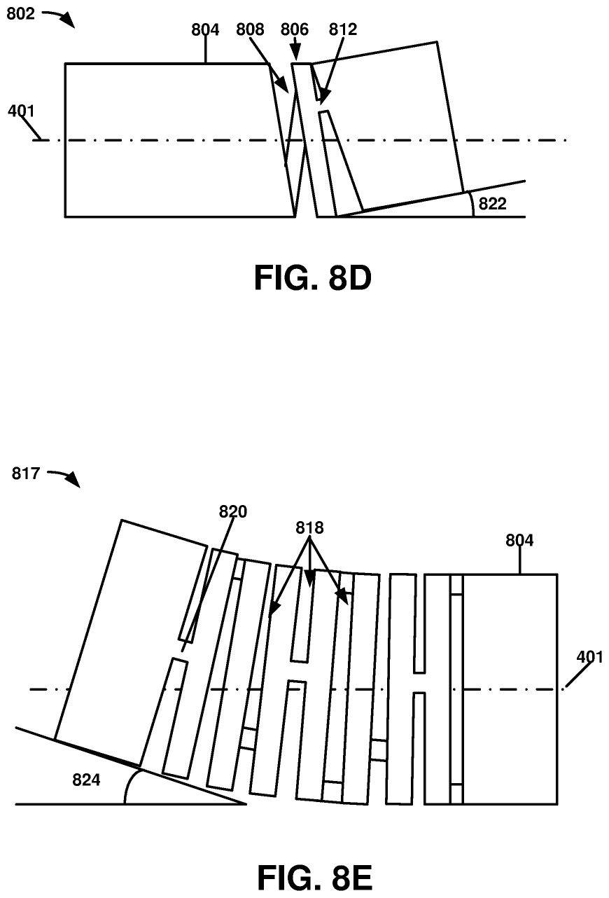

FIG. 8A is a cross-sectional diagram illustrating a cross-sectional view of an example electrode 802 of lead 106 of FIG. 4A, the cross-section being taken along longitudinal axis 401 of lead 106 of FIG. 4A. Electrodes 802 may be disposed on lead 106 in a similar manner to other electrodes (e.g., electrodes 117, 502, 602) as described above. Electrode 802 may include an elongated body 803 (also referred to as “elongated body 803”) extending along longitudinal axis 401 and including a distal portion 804A, a proximal portion 804B, and a medial portion 806 between distal portion 804A and proximal portion 804B. Elongated body 803 may define an inner surface 805A and an outer surface 805B. Inner surface 805A may define lumen 403 of electrode 802.

Medial portion 806 may extend from a longitudinal center of electrode 802. Distal portion 804A and proximal portion 804B may extend from the distal end and the proximal end of medial portion 806, respectively. One or more openings 808 may be formed in medial portion 806.

Openings(s) 808 (alternatively referred to as “cut(s) 808, slot(s) 808”) may be configured to expand and/or contract along a longitudinal length of electrode 802, e.g., in response to movement of tongue 118. In such a manner, the expansion and contraction of opening(s) 808 may facilitate flexure of electrode 802, e.g., about a plane orthogonal to longitudinal axis 401. Flexure of electrode 802 may reduce concentration of stress and/or strain at distal portion 804A and/or proximal portion 804B, thereby reducing wear on the connections between electrode 802 and adjacent flexible portions 402 of lead body 112 (e.g., as illustrated in FIG. 4). In some examples, opening(s) 808 may be or may be connected to or arrange in a similar arrangement as any of openings 510 and/or 608 (e.g., as illustrated in FIGS. 5A-6C).

Opening(s) 808 may define a spiral or coil extending around an outer perimeter of electrode 802 (e.g., along outer surface 805B). In other examples, opening(s) 808 may extend around the outer perimeter of electrode 802 along reference planes orthogonal to longitudinal axis 401. Each of openings 808 may extend partially from outer surface 805B of electrode 802 towards inner surface 805A of electrode 802 or vice versa. In some examples, each of openings 808 may extend from outer surface 805B through to inner surface 805A.

In some examples, a single opening 808 may define the spiral or coil extending around the outer perimeter of electrode 802. In some examples, as illustrated in FIG. 8A, two or more openings 808 may define an interrupted spiral or coil extending around the outer perimeter. The two or more openings 808 may be separated by segments 812 disposed around the outer perimeter. Segments 812 may increase the integrity of electrode 802 and may prevent unintended wear or separation of portions of electrode 802 during the flexure of electrode 802.

Electrode 802 may further include one or more openings 404 (not pictured) extending from the outer surface to the inner surface. Openings 404 may not be configured to expand and/or contract in response to movement of tongue 108. Openings 404 may electrically couple electrode 802 to conductor 406 (not pictured), e.g., as previously described herein.

FIG. 8B is a cross-sectional diagram illustrating a cross-sectional view of example electrode 814 of lead 106 of FIG. 4A, the cross-section being taken along longitudinal axis 401 of lead 106 of FIG. 4A. Electrode 814 may be similar to electrode 802 with the addition of one or more openings 816 disposed around the outer perimeter of electrode 814 and at one or more of the distal portion 804A or proximal portion 804B of electrode 814.

Opening(s) 816 may be similar to openings 808 and may be configured to expand and/or contract along the longitudinal length of electrode 814 in response to movement of tongue 118. The expansion and/or contraction of opening(s) 816 may allow electrode 814 to flex along distal portion 804A and/or proximal portion 804B containing opening(s) 816. The additional flexure at distal portion 804A and/or proximal portion 804B may further reduce stress and/or strain concentration at the corresponding end and may reduce wear and/or separation of the connections between electrode 814 and adjoining flexible portions 402 (not pictured). Electrode 814 may also include one or more openings 404 (not pictured) configured to electrically couple electrode 814 to conductor 406 (not pictured).

FIG. 8C is a cross-sectional diagram illustrating a cross-sectional view of an example electrode 817 of electrodes 117 of lead 106 of FIG. 4A, the cross-section being taken along longitudinal axis 401 of lead 106 of FIG. 4A. Electrodes 817 may be disposed on lead 106 in similar manners as other electrodes (e.g., electrodes 117, 502, 602, 802, 814) as described above. Electrode 817 may include a plurality of openings 818 extending along longitudinal axis 401, each opening 818 extending at least partially around the outer perimeter of electrode 817 (e.g., along outer surface 805B of elongated body 803 of electrode 817) and separated from a circumferentially adjacent opening 818 by segment 820. The plurality of openings 818 may extend from a distal end of electrode 817 to a proximal end of electrode 817. Openings 818 may allow for increased flexure of electrode 817 compared to electrodes 802, 814 and may further reduce stress and strain concentration at the distal and proximal ends of electrode 817. Each of openings 818 may extend at least partially from outer surface 805B to inner surface 805A, at least partially from inner surface 805A to outer surface 805B, or extend entirely from outer surface 805B to inner surface 805A.

In some examples, the flexible material(s) defining flexible portions 402 and/or portions of flexible portions 402 may be disposed within at least some of openings 808, 818, thereby increasing the strength of the bonds between flexible portions 402 and electrode 802, 814, or 817. In some examples, the volumes of space defined by openings 808, 818 may retain another flexible material, e.g., to facilitate increased flexure of electrodes 802, 814, or 817. In some examples, electrodes 802, 814, or 817 may be disposed over a pre-formed lead body 112 and openings 808, 818 may be exposed, e.g., to facilitate flexure of electrodes 802, 814, or 817.

FIG. 8D is a conceptual diagram illustrating flexure of electrode 802 of FIG. 8A. FIG. 8E is a conceptual diagram illustrating flexure of electrode 817 of FIG. 8C. As illustrated in FIGS. 8D, and 8E, electrodes 802, 817 may flex and openings 806, 818 may expand or contract accordingly, depending on the direction of flexure. In some examples, electrodes 802, 817 may be configured to be biased towards particular directions of flexure. In some examples, electrodes 802, 817 may be configured to flex in any direction about longitudinal axis 401.

As illustrated in FIG. 8D, electrode 802 may flex away from longitudinal axis 401 by and angle 822. As illustrate din FIG. 8E, electrode 817 may flex away from longitudinal axis 401 by angle 824. Angle 827 may be greater than angle 822, e.g., due to an increased number of openings 818 disposed along the longitudinal length of electrode 817 than the number of openings 808 disposed along the longitudinal length of electrode 802. When flexed electrodes 802, 817 may define a radius of curvature greater than or equal to 8.38 millimeters (mm) (e.g., about 0.33 inches (in)).

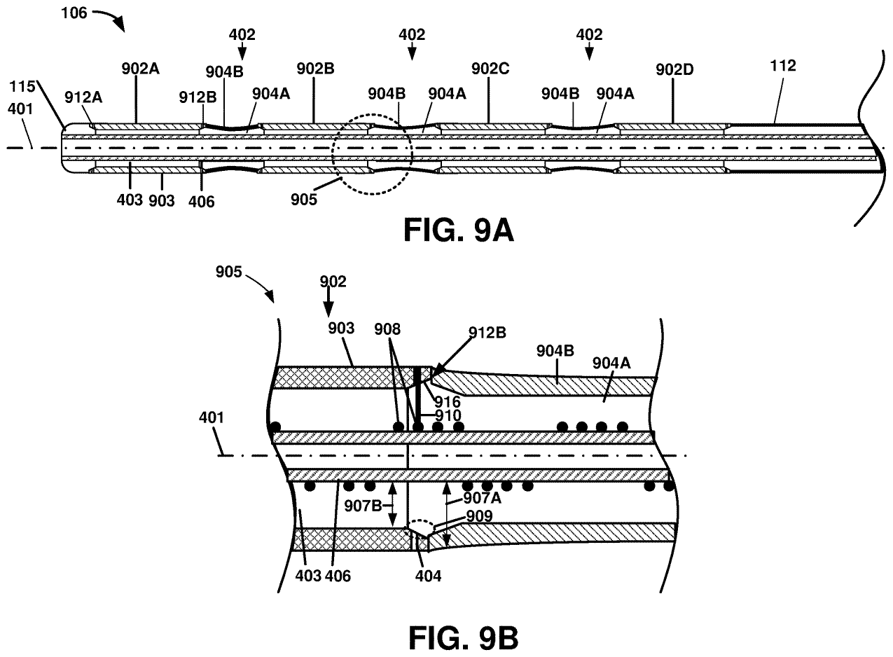

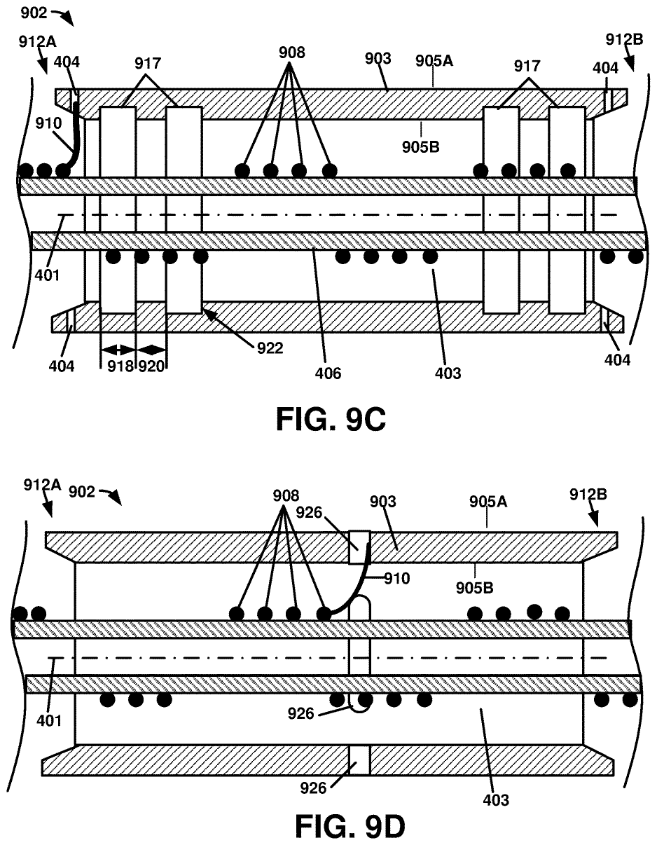

In some examples, FIGS. 8A-8D may describe an implantable lead 106 configured to be placed near a hypoglossal nerve of a patient, comprising: lead body 112 defining a longitudinal axis 401; a conductor coil 406 disposed within an inner lumen 403 of lead body 112 and extending along the longitudinal axis 401, wherein the conductor coil 406 comprises a plurality of conductive wires (e.g., conductive wire 908 as illustrated below in FIG. 9B); and a plurality of electrodes (e.g., electrode 802, 814, 817) disposed on a distal portion of the elongated shaft 112, each electrode comprising: an elongated electrode body 803 extending from a proximal end to a distal end, wherein the elongated electrode body 803 defines: an outer surface 805A and an inner surface 805B in contact with the inner lumen 403, and one or slots (e.g., slots 808, 816, 818) extending at least partially from the outer surface 805A towards the inner lumen 403 and at least partially around a perimeter of the elongated electrode body 803; and an opening (e.g., opening 404, not pictured in FIGS. 8A-E) configured to retain a respective conductive wire of the plurality of conductive wires extending from the conductor coil 406, wherein each electrode of the plurality of electrodes is electrically connected to the conductor coil 406 via the respective conductive wire, and wherein each electrode of the plurality of electrodes is affixed to a first flexible portion (e.g., flexible portion 402, as illustrated in FIG. 4A) at the distal end and to a second flexible portion (e.g., another flexible portion 402) at the proximal end.

FIG. 9A is a cross-sectional diagram illustrating a cross-sectional view of another example of implantable lead 106 of FIG. 4A longitudinal axis 401 of lead 106 of FIG. 4A. Lead 106 may include electrodes 902A-D (collectively referred to as “electrodes 902”) disposed on lead body 112 of lead 106 and along longitudinal axis 401. Longitudinally adjacent electrodes 902 may be separated by flexible portions 402 of lead body 112. Flexible portions 402 may connect to adjoining electrodes 902 at junctions 905. Conductor 406 may be disposed within lead body 112 and extend along longitudinal axis 401 to distal end 115 of lead 106. Each of electrodes 902 may be coupled to conductor 406.

Lead body 112 may be formed from at least two flexible materials 904 including first flexible material 904A and second flexible material 906B. Each of flexible materials 904 may define different stiffness and/or hardness values. For example, first flexible material 904A may have a different durometer hardness value than second flexible material 904B. First flexible material 904A may be disposed around conductor 406 and second flexible material 904B may be disposed around and radially outwards of first flexible material 904A. First flexible material 904A may be less flexible than second flexible material 904B. Disposal of the second flexible material 904B over first flexible material 904A increases flexibility of lead body 112 while maintaining integrity and/or pushability of lead body 112. In some examples, a coating (e.g., an anti-bacterial coating) may be disposed over an outer surface of second flexible material 904B and define an outer surface of lead 106.

Each electrode 902 may include an elongated body 903 extending along longitudinal axis 401 from a proximal end 912B to a distal end 912A. Elongated body 903 may define an outer surface (e.g., defining an outer diameter of lead 106) and an inner surface defining lumen 403.

FIG. 9B is a cross-sectional diagram illustrating a cross-sectional view of an example junction 905 of lead of FIG. 9A, the cross-section being taken along longitudinal axis 401 of lead 106 of FIG. 9A. For each of ends 912A, 912B (collectively referred to as “ends 912”) of electrodes 902, each end 912 may be connected to a flexible portion 402 (e.g., to first flexible material 904A and to second flexible material 904B). As illustrated in FIG. 9B, an inner edge of each of ends 912 of each of electrodes 902 may be chamfered to form chamfer 916. Each electrode 902 may define chamfer 916 at one of ends 912 (e.g., at a distal end 912A, at a proximal end 912B) or at both ends 912.

Chamfer 916 may extend around the entirety of the inner perimeter of electrode 902. Chamfer 916 may increase a surface contact area between electrode 902 and flexible materials 904. For example, as illustrated in FIG. 9B, chamfer 916 increases the contact area between first flexible material 904A and electrode 902. The increase in surface area may increase the strength of the bond between electrode 902 and flexible portion 402 and/or reduce wear on the bond due to the repeated application of stress and/or strain. Chamfer 916 may allow for a relatively more gradual transition between different thickness of flexible materials 904, e.g., as compared to another electrode 902 without chamfer 916. Flexible materials 904 may define a first thickness 907A within flexible portions 402 and a second thickness 907B within inner lumen 403 of electrodes 902. By defining a more gradual transition between first thickness 907A and second thickness 907B, electrode 902 may reduce a stress concentration at a transition region 909 between first thickness 907A and the second thickness 907B and thereby reduce the stress concentration at the junction between flexible portion 402 and electrode 902.

Each chamfer 916 may define a corresponding chamfer angle. Chamfer angle may be an angle offset from longitudinal axis 410 and defining the angle of chamfer 916 relative to longitudinal axis 401. In some examples, where electrode 902 defines two chamfers 916, one at each end of electrode 902, each chamfer 916 may define a different chamfer angle.

As discussed previously herein, electrode 902 may include one or more openings 404 extending from the outer surface to the inner surface. In some examples, openings 404 may extend from the outer surface to the chamfered surface defining chamfer 916. Each of openings 404 may retain an intermediate conductive wire 910 connecting electrode 902 to a corresponding conductive wire 908 of conductor 406. Intermediate conductive wire 910 may be affixed to electrode 902 (e.g., within opening 404 via welding, crimping, or other techniques.

FIG. 9C is a cross-sectional diagram illustrating a cross-sectional view of another example of electrode 902 of FIG. 9A, the cross-section being taken along longitudinal axis 401 of lead 106 of FIG. 9A. As illustrated in FIG. 9C, electrode 902 may define a one or more grooves 917 extending from inner surface 905B of elongated body 903 of electrode 902 partially towards outer surface 905A of elongated body 903 of electrode 902. Grooves 917 may be present on electrode 902 instead of or in addition to chamfers 916. Grooves 917 may be disposed on certain portions of electrode 902, e.g., on a distal portion and/or a proximal portion of electrode 902, as illustrated in FIG. 9C. In some examples, grooves 917 may be disposed along an entire longitudinal length of elongated body 903 of electrode 902.

Each groove 917 may define a width 918 and a depth 920. Longitudinally adjacent grooves 917 may be separated by a distance 920. Width 918, depth 922, and distance 920 may be the same for all grooves 917 on a single electrode 902 or may vary between grooves 917 on electrode 902. Each groove 917 may extend partially or entirely around an inner perimeter of electrode 902 (e.g., along inner surface 905B of elongated body 903). Each groove 917 may be configured to retain flexible material(s) of flexible portions 402 and/or connected to flexible portions 402 (not pictured) including, but is not limited to, first flexible material 904A and/or second flexible material 904B. Placement of flexible material(s) within grooves 917 may increase the contact surface area between electrode 902 and flexible portions 402. The increased contact surface area may strengthen a mechanical interlock between flexible materials 904 of flexible portions 402 and the material of electrode 902 (e.g., the material defining walls of grooves 917). The increased strength in the mechanical interlock may reduce motion of flexible materials 904 and/or conductor 406 along longitudinal axis 401 and thereby reduce strain on flexible materials 904 and/or conductor 406.

Openings 404 may retain intermediate conductive wire 910 to couple electrode 902 to conductive wire 908 of conductor 406. For example, intermediate conductive wire 910 may couple outer surface 905A of elongated body 903 of electrode 902 to conductive wire 908. Openings 404 may be disposed at locations along the longitudinal length of electrode 902. In some examples, as illustrated in FIG. 9B, openings 404 may be disposed at locations distal to a distal-most groove 917 and/or proximal to a proximal-most groove 917. In some examples, openings 404 may be disposed between two grooves 917 and/or within a groove 917. Placement of openings 404 closer to a longitudinal center of electrode 902 may reduce the strain and/or stress applied on intermediate conductive wire 910, thereby reducing the wear on a bond between intermediate conductive wire 910 and electrode 902 and/or conductive wire 908.

In some examples, implantable lead 106 is configured to be placed near a hypoglossal nerve of a patient, lead 106 comprising: an lead body 112 defining a longitudinal axis 401; a conductor coil 406 disposed within an inner lumen 403 within of the lead body 112 (e.g., as defined by inner lumen 403 of electrodes 902 and extending along longitudinal axis 401, wherein the conductor coil comprises a plurality of conductive wires 908; and a plurality of electrodes 902 disposed on a distal portion of lead body 112, each electrode 902 comprising: an elongated electrode body 903 extending from a proximal end 912B to a distal end (e.g., the other of ends 912), wherein elongated electrode body 903 defines an outer surface 905A and an inner surface 905B in contact with inner lumen 403 (e.g., inner surface 905B defining inner lumen 403), and wherein elongated electrode body 903 defines one or more grooves 917 extending from inner lumen 403 towards outer surface 920A; an opening 404 configured to retain a respective conductive wire 910 of the plurality of conductive wires 910 extending from conductor coil 406, wherein each electrode 902 of the plurality of electrodes 902 is electrically connected to conductor coil 406 via the respective conductive wire 910, and wherein each electrode 902 of the plurality of electrodes 902 is affixed to a first flexible portion 402 at distal end 912A and to a second flexible portion 402 at proximal end 912B.

FIG. 9D is a cross-sectional diagram illustrating a cross-sectional view of another example of electrode 902 of lead 106 of FIG. 9A, the cross-section being taken along longitudinal axis 401 of lead 106 of FIG. 9A. As illustrated in FIG. 9D, electrode 902 may define a plurality of openings 926 disposed around an outer perimeter of electrode 902 (e.g., along outer surface 905A of elongated body 903 of electrode 902). Each opening 926 may extend from outer surface 905A to inner surface 905B. The example electrode 902 of FIG. 9D may define openings 926 instead of or in addition to one or more grooves 916 and/or one or more chamfers 916.

Each opening 926 may define an elongated slot extending along a plane orthogonal to longitudinal axis 401. Openings 926 may be configured to be placed at or near a longitudinal center of electrode 902 (i.e., a longitudinal center of elongated body 903 of electrode 902). Each opening 926 may be configured to retain intermediate conductive wire 910, e.g., similar to openings 404 of any of FIGS. 1-9B. As compared to openings 404, the locations of openings 906 at or near the longitudinal center of electrode 902 may reduce magnitude of the stress and/or strain applied to intermediate conductive wire 910 and/or to a connection between intermediate conductive wire 910 and electrode 902, e.g., as a result of flexure of tongue 118. The decreased stress and/or strain may reduce wear and a likelihood of unintended separation of intermediate conductive wire 910 from at least one of electrode 902 or conductive wire 908 of conductor 406.

In some examples, intermediate conductive wire 910 may only be disposed within one of a plurality of openings 926. In such examples, the other openings 926 may be configured to retain flexible material(s) connected to flexible portions 402 (e.g., first flexible material 904A, second flexible material 904B). Retention of the flexible material(s) may increase the contact surface area between electrode 902 and flexible portions 402, thereby increasing the bond strength between electrode 902 and flexible portions 402 and/or forming additional bonds between electrode 902 and flexible portions 402.

In some examples, as illustrated in FIG. 9D, openings 926 may be equally distributed around the entire outer perimeter of electrode 902. In some examples, openings 926 may be restricted to a single portion of the outer perimeter of electrode 902. Each of electrodes 902 may include two or more openings 926.

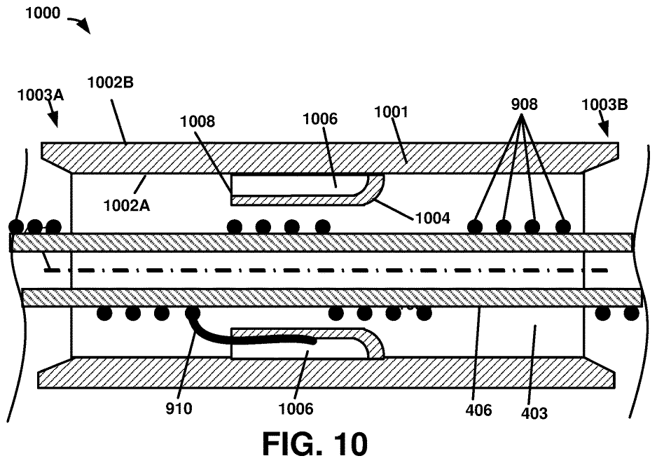

FIG. 10 is a cross-sectional diagram illustrating a cross-sectional view of another example electrode 1000 of lead 106 of FIG. 4A, the cross-section being taken along longitudinal axis 401 of lead 106 of FIG. 4A. Electrode 1000 may an example electrode configuration for electrode 117 disposed on lead 106 (e.g., as illustrated in any of FIGS. 4A-9D). As illustrated in FIG. 10, electrode 1000 may include elongated body 1001 defining an inner surface 1002A and an outer surface 1002B. Inner surface 1002A may define inner lumen 403 of electrode 1000. Elongated body 1001 may include one or more protrusions 1004 extending (e.g., radially inwards towards longitudinal axis 401) from inner surface 1002A of electrode 106. Protrusions 1004, in conjunction with inner surface 1002A, may define one or more channels 1006.

Each channel 1006 may be configured to retain intermediate conductive wire 910. Intermediate conductive wire 910 may be affixed to electrode 1000 within channel 1006 (e.g., to inner surface 1002, to protrusion 1004). For example, intermediate conductive wire 910 may be affixed to inner surface 1002A of elongated body 1001. Elongated body 1001 may be entirely electrically conductive. Intermediate conductive wire 910 may deliver electrical stimulation signals to inner surface 1002A and/or protrusion 1004. Inner surface 1002A and/or protrusion 1004 may then transmit the electrical stimulation signals to outer surface 1002B of electrode 1000. Outer surface 1002B of electrode 1000 may then transmit the electrical stimulation signals to tissue of tongue 108. Placement of intermediate conductive wire 910 within channels 1006 may reduce a magnitude of stress and/or strain applied to intermediate conductive wire 910, to the bonds between intermediate conductive wire 910 and electrode 1000 and/or to the bonds between conductive wire 908 of conductor 406.

Each channel 1006 may define an opening 1008 at one end. In some examples, as illustrated in FIG. 10, channel 1006 may be enclosed at an end opposite opening 1008. In such examples, channels 1006 may extend from openings 1008 towards a distal end 1003A or a proximal end 1003B of electrode 1000. In some examples, one or more channels 1006 may extend from corresponding openings 1008 towards proximal end 1003B and one or more other channels 1006 may extend from corresponding openings 1008 towards distal end 1003A. In some examples, each channel 1006 may define openings 1008 at both ends. For each channel 1006, one of the corresponding openings 1008 may be disposed at or near a longitudinal center of elongated body 1001.

Each channel 1006 may define a substantially circular cross section. In some examples, channel 1006 may define an inner diameter configured to retain intermediate conductive wire 910. The inner diameter may be about 0.089 mm to about 0.12 mm (e.g., about 0.0035 in to about 0.0045 in). In some examples, portions of inner surface 1002A may define a groove that when aligned with corresponding curvature of protrusion 1004, defines the cross-sectional profile of channel 10006. Protrusions 1004 may be a same or different material as elongated body 1001. For example, each of elongated body 1001 and protrusions 1004 may be formed from an Titanium alloy (e.g., a Titanium Molybdenum alloy), a Tantalum alloy, or a Tin alloy. Protrusions 1004 may be integral to elongated body 1001 and may be formed from elongated body 1001 as a part of the manufacturing process of electrode 1000. In some examples, protrusions 1004 may be separate components and may be affixed to inner surface 1002A (e.g., via welding) after manufacturer of electrode 1000.

As compared to the use of openings (e.g., openings 404, 926) to retain an intermediate conductive wire, use of channels 10006 to retain conductive wire may further reduce stress and/or strain applied to the bonds between intermediate conductive wire 910 and electrode 1000 and/or conductor 406, thereby reducing wear on the bonds and/or unintended separation of intermediate conductive wire 910 from electrode 1000 and/or conductor 406.

In some examples, FIG. 10 may illustrate an implantable lead 106 configured to be placed near a hypoglossal nerve of a patient, comprising: an lead body 112 defining a longitudinal axis 401; a conductor coil 406 disposed within an inner lumen 403 of the elongated shaft and extending along the longitudinal axis 401, wherein the conductor coil 406 comprises a plurality of conductive wires 908; and a plurality of electrodes 1000 disposed on a distal portion of the lead body 112, each electrode 1000 comprising: an elongated electrode body 1001 extending from a proximal end 1003B to a distal end 1003A, wherein the elongated electrode body 1001 defines an outer surface 1002B and an inner surface 1002A in contact with the inner lumen 403; and a channel 1006 disposed on the inner surface 1002A of the elongated electrode body 1001, wherein the channel 1006 is at least partially enclosed, and wherein the channel 1006 is configured to retain a respective conductive wire 908 of the plurality of conductive wires 908 extending from the conductor coil 406, wherein each electrode 1000 of the plurality of electrodes 1000 is electrically connected to the conductor coil 406 via the respective conductive wire 908, and wherein each electrode 1000 of the plurality of electrodes 1000 is affixed to a first flexible portion 402 at the distal end 1003A and to a second flexible portion 402 at the proximal end 1003B.

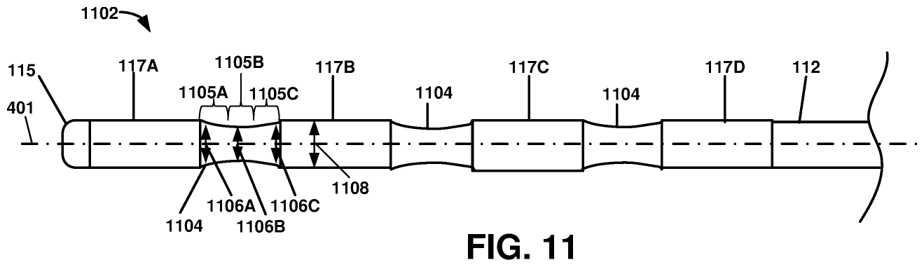

FIG. 11 is a conceptual diagram illustrating another example implantable lead 106 of FIG. 1. Lead 106 of FIG. 11 may define a distal portion 1102 of lead body 112. Distal portion 1102 may include electrodes 117 separated by flexible portions 1104. Each flexible portion 1104 may define a plurality of regions 1105 A-C (collectively referred to as “regions 1105”). Regions 1105 may include a distal region 1105A, a medial region 1105B proximal to distal region 1105A, and a proximal region 1105C proximal to medial region 1105B. In other examples, flexible portion 1104 may define two regions 1105 or four or more regions 1105. Each of regions 1105 may define a corresponding outer diameter. For example, distal region 1105A defines outer diameter 1106A, medial region 1105B defines outer diameter 1106B, and proximal region 1105C defines outer diameter 1106C. Flexible portion 1104 may define a reduction in outer diameter from an outer diameter 1108 of electrodes 117 to a minimum outer diameter of flexible portion 1104 (e.g., outer diameter 1106B). The reduction in the outer diameter of flexible portion 1104 may increase the concentration or stress and/or strain along distal portion 1102 to locations along flexible portions 1104 defining minimum outer diameter 1106 (e.g., to medial portion 1105B of flexible portion 1104), thereby reducing stress and/or strain on the junctions between electrodes 117 and flexible portions 1104.

In some examples, medial portion 1105B may define a concave shape terminating in a point defining outer diameter 1106B. Outer diameter 1106A may be the same as one or more of outer diameter 1106C or outer diameter 1108 of electrode 117, or vice versa. Each of outer diameter 1106A and outer diameter 1106C may be greater than outer diameter 1106B, e.g., to cause medial potrion 1105B to define a concave shape.

In some examples, as illustrated in FIG. 11, medial portion 1105B may encompass a longitudinal center of flexible portion 1104, e.g., such that flexible portion 1104 defines a symmetrical structure along longitudinal axis 401. In some examples, at least a portion of medial portion 1105B (e.g., a portion defining outer diameter 1106B) may be distal to or proximal to the longitudinal center of flexible portion 1104. In some examples, medial portion 1105B may be relatively narrow along longitudinal axis 401, e.g., as to define a groove along flexile portion 1104 defining outer diameter 1105B.

Each flexible portion 1104 may include one or more flexible material(s), e.g., as similar to flexible portions 402 of any of FIGS. 1-10. Flexible portion 1104 may define a reduction in outer diameter towards a specific point along the longitudinal length of flexible portion 1104. The specified point may be along a longitudinal center of flexible portion 1104, e.g., such that flexible portion 1104 defines a symmetrical, concave curvature from one end to the other. In such examples, distal portion 1105A, medial portion 1105B, and proximal portion 1105C may each define a same longitudinal length. In some examples, distal portion 1105A and proximal portion 1105C may define a same longitudinal length. In some examples, regions 1105 define a biased concave curvature towards a distal end or a proximal end of flexible portion 1104. In such examples, distal portion 1105A, medial portion 1105B, and proximal portion 1105C may each define different longitudinal lengths.

In some examples, the reduction in the outer diameter of flexible portion 1104 may extend along the entire longitudinal length or substantially the entire longitudinal length of flexible portion 1104, e.g., as illustrated in FIG. 11. In some examples, the reduction in the outer diameter of flexible portion 1104 may only extend along a specific portion of flexible portion 1104 (e.g., around the specified point The change in the outer diameter between two of outer diameters 1106A, 1106B, 1106C, and outer diameter 1108 of electrode 117 may be continuous or stepwise. A continuous change in the outer diameter of lead 106 may represent a constant change and/or slope between the two outer diameters. A stepwise change in the outer diameter of lead 106 may represent one or more discrete changes in the outer diameter from one outer diameter value to the other outer diameter value. Outer diameter 1108 of electrode 1000 may be less than or equal to about 1.27 mm (e.g., about 0.005 in). For example, outer diameter 1108 may be about 1.02 mm to about 1.27 mm. Minimum outer diameter 1106B may be about 0.91 mm (e.g., about 0.0036 in) to about 1.02 mm. The stepwise change in the outer diameter of lead 1106 along the length of flexible portion 1104 may facilitate preferential bending of lead 106 away from electrodes 117, e.g., thereby reducing stress and strain on connection points between ends of electrodes 117 and flexible portions 1104.

In some examples, this disclosure describes an implantable lead 106 configured to be placed near a hypoglossal nerve of a patient, comprising: an lead body 112 defining a longitudinal axis 401 and an inner lumen (inner lumen 403 of FIG. 4A), wherein the lead body 112 comprises: a plurality of electrodes 117 disposed on the lead body 112; a plurality of flexible regions 1104, each flexible region 1104 defining a concave cylindrical shape along the longitudinal axis 401, wherein each flexible region 1104 separates longitudinally adjacent electrodes 117 of the plurality of electrodes 117, and wherein the concave cylindrical shape comprises: a distal portion 1105A defining a first diameter 1106A; a proximal portion 1105C defining a second diameter 1105C; and a concave portion 1105B connecting the distal portion 1106A and the proximal portion 1106C and defining a third diameter 1106B at a narrowest location along the concave portion 1105B, wherein the third diameter 1106B is less than the first diameter 1106A or the second diameter 1106C; a conductor coil (e.g., conductor 406 of FIG. 4A) disposed within the inner lumen and extending along the longitudinal axis 401, wherein the conductor coil comprises a plurality of conductive wires (e.g., conductive wires 908 of FIG. 9B), and wherein each electrode 117 of the plurality of electrodes 117 is electrically connected to a respective wire of the plurality of conductive wires.

In some examples, this disclosure describes an implantable lead 106 configured to be placed near a hypoglossal nerve of a patient, comprising: an lead body 112 defining a longitudinal axis 401 and comprising: a plurality of electrodes 117; and a plurality of separate flexible regions 1104 arranged along the longitudinal axis 401, each flexible region 1104 extending along the longitudinal axis 401 from a proximal end to a distal end and comprising: a first portion 1105A defining a first outer diameter 1106A; a second portion 1105C defining a second outer diameter 1106C; and a third portion 1105B connected to the first portion 1105A and the second portion 1105C and defining a third outer diameter 1106B, wherein the third outer diameter 1106B is less than the first outer diameter 1106A or second outer diameter 1106C.

While FIGS. 4A-11 illustrate different designs for lead 106 (e.g., different electrode designs, different flexible region designs). An example lead 106 described in this disclosure may include any combination of the features described herein. For example, each of electrodes 117 may be one of or a combination of features of electrodes 502, 602, 702, 802, 814, 817, 902, or 1000. Electrode features may include, but are not limited to, junctions 504, 604, openings 404, 510, 608, 808, 818, 926 segments 812, chamfers 916, grooves 917, channels 1006, conductive wires 704, 910, or any combination thereof. Similarly, flexible portions 402 of lead 106 may include flexible portions 1104.

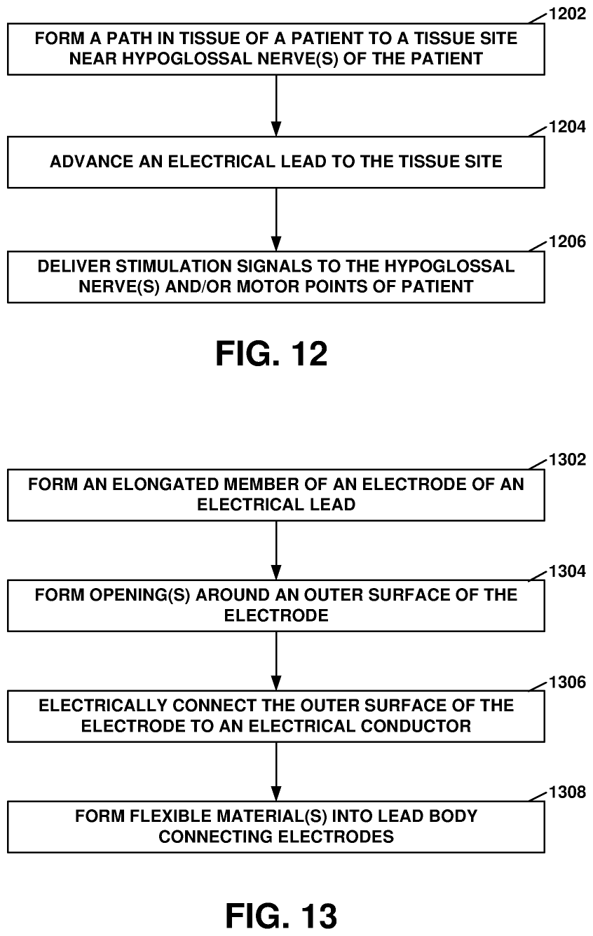

FIG. 12 is a flow diagram illustrating an example process for implanting an implantable lead in tissue of a patient. While FIG. 12 is primarily described with reference to lead 106 and electrode 117 of FIG. 4A, the example process may be performed with any other example leads and electrodes described herein. For example, the lead may be one or more of or a combination of the features of any of leads 106 illustrated in FIGS. 5A-11. Similarly, electrodes disposed on lead 106 may be any one or more of or a combination of the features of any of electrodes 502, 602, 702, 802, 814, 817, 902, or 1000, as previously described herein.

A clinician may use a needle of a medical device system 100 to create path in tissue of patient 102 to a target area near hypoglossal nerve(s) of patient 102 (1202). The clinician may insert a needle of system 100 through tissue near a chin of patient 102 and through tongue of patient 102 to the target area. The target area may be within one or more of protrusor muscles 120 and/or 122 and near one or more hypoglossal nerve(s) and/or motor points. During insertion of the needle into tissue of patient 102, the clinician may insert a trocar into a needle lumen defined by the needle to control an amount of bodily fluids (e.g., blood) in the path in the tissue.

The clinician may deliver test electrical signals (e.g., test stimulation signals) via one or more electrodes on the needle and sense electrical signals from tissue of patient 102 (e.g., evoked electrical signals) via the one or more electrodes on the needle. Based on the sensed electrical signals, the clinician may determine whether the needle is properly positioned within the tissue of patient 102 and readjust the needle within the tissue if the clinician determines that the needle is not properly positioned. The clinician may iteratively deliver test electrical signals to the tissue of patient 102, sense electrical signals from the tissue, and reposition the needle within the tissue until the clinician determines that the needle is properly positioned within the tissue of patient 102.

The clinician may navigate introducer through the path to the target area near hypoglossal nerve(s) of patient 102. The clinician may retract the needle from the path to the target area and then advance the introducer to through the path to the target area. In some examples, the clinician may advance a guide member (e.g., a guidewire) through a needle lumen of the needle to the target area, retract the needle proximally, and advance the introducer along the guide member to the target area. Once the introducer is advanced to the target area, the clinician may retract guide member proximally from the introducer lumen.

The clinician may advance lead 106 to the target area near hypoglossal nerve(s) of patient 102 (1204). The introducer defines an introducer lumen extending from a distal end of the introducer to a proximal end of the introducer. The clinician may dispose lead 106 into the introducer lumen through the proximal end of the introducer and advance lead 106 along the introducer lumen until electrodes 117 disposed on a distal portion of lead 106 are at the target area near hypoglossal nerve(s) and/or motor points of patient 102. When electrodes 117 of lead 106 are at the target area, distal portion of lead 106 may extend distally from the distal end of the introducer.

The clinician may deliver, via one or more of electrodes 117, test stimulation signals to tissue of patient 102 and sense electrical signals from the tissue of patient 102 in response to the test stimulation signals. Based on the sensed electrical signals, the clinician may determine that one or more of electrodes 117 are improperly positioned within the tissue of patient 102, reposition lead 106 within the tissue of patient 102 to a proper position within the tissue of patient 102.

Once the clinician determines that electrodes 117 are properly positioned within the tissue of patient 102, the clinician may deploy one or more fixation elements 116 disposed on lead 106 (e.g., fixation tines, fixation helixes and/or coils) to affix lead 106 within tissue of patient 102 at the proper position.

System 100 may deliver, via one or more of electrodes 117, electrical stimulation signals to the hypoglossal nerve(s) and/or motor points of patient 102 (1206). System 100 may determine whether patient 102 is experiencing occlusive sleep apnea and/or whether tongue 118 is occluding or constricting the upper airway of patient 102. System 100 may then deliver electrical stimulation signals to the hypoglossal nerve(s) and/or motor points of patient 102 to cause tongue 118 to protrude, e.g., to cause movement of tongue 118 and open up the upper airway of patient 102.

Movement of tongue 118 may include flexure of tongue 118 and may cause corresponding flexure of lead 106 implanted within tongue 118. Each flexure of tongue 118 may cause concentration of stress and/or strain at specific locations along lead 106 (e.g., at junctions between electrodes 117 and flexible portions (e.g., flexible portions 402, 1104) of lead body 112. In addition, other movement of tongue 118 (e.g., when patient 102 is speaking, eating, or the like) may cause similar flexure of lead 106 and similar concentrations of stress and/or strain at the specific locations.

Electrodes 117 and/or flexible portions of lead body 112 may include one or more features configured to distribute the stress and/or strain applied to lead 106 and reduce wear and/or likelihood of separation at the junctions between electrodes 117 and the flexible portions of lead body 112. The features may include, but are not limited to, junctions 504, 604, openings 404, 510, 608, 808, 818, 926, segments 812, chamfers 916, grooves 917, channels 1006, conductive wires 704, 910, or any combination thereof. The features may reduce wear on bonds between electrodes 117 and lead body 112 and/or conductor 406 disposed within lead body 112 caused by the repeated applications of stress and/or stain and may increase the operational lifespan of lead 106. In some examples, the features may increase flexibility of lead 106, e.g., by increasing flexure capabilities of electrodes.

FIG. 13 is a flow diagram illustrating an example process for manufacturing an example implantable lead. While the example process illustrated in FIG. 13 is described primarily with reference to electrode 117 and lead 106, the example process may be applied or modified to manufacture other electrodes described in this disclosure.

A manufacturer may form an elongated body of an electrode 117 of an electrical lead 106 (1302). Elongated body may include any of elongated body 506, 606, 803, 903, or 1001. A manufacturer may remove, from a sheet of electrically conductive material, a portion of the electrically conductive material having an area greater than or equal to an intended surface area of the elongated body. The manufacturer may then shape the portion of electrically conductive material into the elongated body. In some examples, the manufacturer may wrap the portion of electrically conductive material around a mandril to form the elongated body. Elongated body may define an elongated annulus extending along longitudinal axis 401 of lead 106. An elongated annulus may include an elongated shape having an outer surface and an inner surface defining a lumen or opening extending through the elongated shape. The manufacturer may then affix circumferentially adjacent ends of the elongated body to form a uniform body (e.g., an elongated annulus). In some examples, the manufacturer may define an electrically active region on an outer surface (e.g., outer surface 514, 805B, 905A, or 1002B) of the elongated body of electrode 117 by disposing electrically conductive or electrically insulative coatings over portions of the outer surface.

In some examples, such as with electrodes 702, the manufacturer may form an electrically conductive material into an elongated wire. The manufacturer may then wrap the elongated wire around mandrill to shape the elongated wire into electrode 702. In some examples, such as with electrodes 1000, where the electrode defines an inner channel (e.g., channel 1006), the manufacturer may form channels 1006 by removing material from the portion of electrically conductive material and/or by affixing a protrusion (e.g., protrusion 1004) to the electrically conductive material before or after shaping the electrically conductive material into the elongated body.

The manufacturer may form opening(s) around an outer surface of electrode 117 (1304). Opening(s) may include, but are not limited to, openings 404, 510, 608, 808, 818, and/or 926. For each opening, the manufacturer may determine the dimensions of the opening (the length, the width, the depth of each opening). For openings 510, 608, 808, 818, and 826, the manufacture may further determine a pattern of openings around the outer surface of electrode 117. The patterns may include equal spacing around a perimeter of electrode 117, a spiral and/or coil pattern extending along the longitudinal length of electrode 117, and/or parallel circular cuts extending around the perimeter of electrode 117. The manufacturer may then remove material from electrode 117 (e.g., via a laser cutting technique, a tool cutting, technique, or the like) based on the dimensions and patterns of openings to define the openings on electrode 117.

In some examples, in addition to or instead of forming openings, the manufacturer may determine dimensions and/or patterns of features including chamfers (e.g., chamfers 916), grooves 917, or the like and form such features onto electrode 117.

The manufacturer may electrically connect the outer surface of electrode 117 to an electrical conductor (e.g., electrical conductor 406) (1306). In some examples, the manufacturer may dispose conductor 406 within inner lumens of electrodes 117. The manufacturer may correspond each of electrodes 117 to a respective conductive wire (e.g., conductive wire 908) on conductor 406. Conductor 406 may define an elongated body with a plurality of conductive wires 908 wrapped around the elongated body and extending along longitudinal axis 401. In some examples, the plurality of conductive wires 908 may form a conductive coil. The manufacturer may then electrically couple each of electrodes 117 to the corresponding conductive wire 908.

In some examples, the manufacturer may dispose an intermediate conductive wire (e.g., intermediate conductive wire 910) within one or more openings on electrode 117. In some examples, the manufacturer may dispose intermediate conductive wire 910 within an channel 1006 on the inner surface of electrode 117. Intermediate conductive wire 910 may contact both electrically conductive portions of electrode 117 and the corresponding conductive wire 908 and may transmit electrical signals between electrode 117 and conductive wire 908. The manufacturer may affix intermediate conductive wire 910 to electrode 117 and conductive wire 908 via one or more techniques such as welding or crimping. For example, the manufacturer may weld one end of intermediate conductive wire 910 to an inner surface of an opening (e.g., opening 404) on electrode 117.

The manufacturer may form flexible material(s) into lead body 112 connecting electrodes 117 (1308). The manufacturer may re-flow one or more flexible materials (e.g., one or more biocompatible polymers to form lead body 112. The flexible materials may be disposed around conductor 406, within an inner lumen defined by electrode 117, within openings on electrode 117, and/or between longitudinally adjacent electrodes 117 such that the material, when solidified, forms a continuous elongated lead body 112. When solidified, the flexible material(s) may define flexible portions (e.g., flexible portions 402) between longitudinally adjacent electrodes 117. At each junction between a flexible portion 402 and an electrode 117, the flexible material(s) may contact electrode 117 along a contact surface area and may form a bond along the contact surface area. Several designs described herein (e.g., chamfers, openings, grooves) may increase the contact surface area and increase the strength of bonds formed therein. In some examples, the manufacturer may reflow a first flexible material 904A around and/or within conductor 406 and electrodes 117 and a second flexible material 904B having different material properties over the first flexible material 904A. Re-flowing two or more flexible materials with different material properties may optimize the mechanical properties of lead 106 and may increase flexibility of lead 106 without compromising the pushability and/or integrity of lead 106.

In some examples, in accordance with the example process of FIG. 13, A method of manufacturing an implantable lead 106 configured to be placed near a hypoglossal nerve of a patient may comprise: forming an electrically conductive material into a plurality of electrodes 117 (1302), each electrode 117 of the plurality of electrodes 117 defining an elongated body (e.g., elongated body 506, 606, 803, 903, or 1001) configured to extend along a longitudinal axis 401 of the lead 106; and forming an lead body 112 of the implantable lead 106 along a longitudinal axis 401 (1308), the lead body 112 comprising: the plurality of electrodes 117; and a plurality of flexible regions 1104 connecting the plurality of electrodes 117, each flexible region 1104 of the plurality of flexible regions 1104 being configured to be disposed between longitudinally adjacent electrodes 117 of the plurality of electrodes 117, and wherein each flexible region 1104 comprises: a distal portion 1105A defining a first diameter 1106A; a proximal portion 1105C defining a second diameter 1106C; and a medial portion 1105B connecting the distal portion 1105A and the proximal portion 1105C and defining a third diameter 1106B at a narrowest location along a longitudinal length of the flexible region 1104, wherein each flexible region 1104 defines a concave cylindrical shape along the longitudinal length of the flexible region 1104.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

It should be noted that system 100, and the techniques described herein, may not be limited to treatment or monitoring of a human patient. In alternative examples, system 100 may be implemented in non-human patients, e.g., primates, canines, equines, pigs, and felines. These other animals may undergo clinical or research therapies that my benefit from the subject matter of this disclosure. Various examples are described herein, such as the following examples.

The following examples are example systems, devices, and methods described herein.

### Example 1

An implantable lead configured to be placed near a nerve of a patient, comprising: an elongated shaft defining a longitudinal axis and an inner lumen, wherein the elongated shaft comprises: a plurality of electrodes disposed on the elongated shaft; a plurality of flexible regions, each flexible region defining a concave cylindrical shape along the longitudinal axis, wherein each flexible region separates longitudinally adjacent electrodes of the plurality of electrodes, and wherein the concave cylindrical shape comprises: a distal portion defining a first diameter; a proximal portion defining a second diameter; and a concave portion connecting the distal portion and the proximal portion and defining a third diameter at a narrowest location along the concave portion, wherein the third diameter is less than the first diameter or the second diameter; and a 52 conductor coil disposed within the inner lumen and extending along the longitudinal axis, wherein the conductor coil comprises a plurality of conductive wires, and wherein each electrode of the plurality of electrodes is electrically connected to a respective wire of the plurality of conductive wires.

### Example 2

The implantable lead of example 1, wherein each flexible region of the plurality of flexible regions comprises one or more polymers.

### Example 3

The implantable lead of example 1, wherein each flexible region comprises a first polymer and a second polymer, and wherein the first polymer defines a different durometer hardness than the second polymer.

### Example 4

The implantable lead of example 3, wherein the first polymer is disposed radially inwards of the second polymer, and wherein the first polymer defines a smaller durometer hardness than the second polymer.

### Example 5

The implantable lead of any of examples 3 and 4, wherein the first polymer defines a first durometer, wherein the second polymer defines a second durometer, and wherein one or more of the first durometer and the second durometer is between 50 durometers and 75 durometers.

### Example 6

The implantable lead of any of examples 3-5, wherein the first durometer is greater than the second durometer.

### Example 7

The implantable lead of example 6, wherein the first diameter is equal to the second diameter.

### Example 8

The implantable lead of any of examples 6 and 7, wherein one or more of the first diameter or the second diameter is about 1.02 millimeters (mm) to about 1.27 mm.

### Example 9

The implantable lead of any of examples 6-8, wherein the third diameter is about 0.91 mm to about 1.02 mm.

### Example 10

The implantable lead of any of examples 1-9, wherein one or more flexible regions of the plurality of flexible regions is symmetrical along the longitudinal axis.

### Example 11

The implantable lead of any of examples 6-9, wherein a first length of the distal portion along the longitudinal axis is greater than a second length of the proximal portion along the longitudinal axis.

### Example 12

The implantable lead of any of examples 6-9, wherein a first length of the distal portion along the longitudinal axis is less than a second length of the proximal portion along the longitudinal axis.

### Example 13

The implantable lead of any of examples 1-12, wherein, for each flexible region, the concave cylindrical shape defines a continuous curvature along the longitudinal axis.

### Example 14

The implantable lead of any of examples 1-13, wherein, for each flexible region, the concave cylindrical shape defines a stepwise change in an outer diameter of the flexible region along the longitudinal axis.

### Example 15

The implantable lead of any of examples 1-14, wherein each electrode of the plurality of electrode defines an elongated annulus extending along the longitudinal axis, wherein each electrode defines an opening disposed on the elongated annulus, and wherein each electrode is electrically connected to the conductor coil via a conductive wire secured within the opening.

### Example 16

The implantable lead of any of examples 1-15, wherein, for each flexible region, the concave cylindrical shape increases strain at a concave portion and reduces strain at a distal end and a proximal end of the flexible region in response to application of a bending force on the lead along a plane normal to the longitudinal axis.

### Example 17

An implantable lead configured to be placed near a nerve of a patient, comprising: an elongated shaft defining a longitudinal axis and comprising: a plurality of electrodes; and a plurality of separate flexible regions arranged along the longitudinal axis, each flexible region extending along the longitudinal axis from a proximal end to a distal end and comprising: a first portion defining a first outer diameter; a second portion defining a second outer diameter; and a third portion connected to the first portion and the second portion and defining a third outer diameter, wherein the third outer diameter is less than the first outer diameter or second outer diameter.

### Example 18

The implantable lead of example 17, wherein each flexible region defines a first step between the first portion and the third potion and a second step between the second portion and the third portion, wherein the first step defines a first change in an outer diameter of the respective flexible region, and wherein the second step defines a second change in an outer diameter of the respective flexible region.

### Example 19

The implantable lead of any of examples 17 and 18, wherein the first outer diameter is equal to the second outer diameter.

### Example 20

The implantable lead of any of examples 17 and 18, wherein the first outer diameter is different from the second outer diameter.

### Example 21

The implantable lead of any of examples 17-20, wherein each flexible region of the plurality of flexible regions comprises one or more polymers.

### Example 22

The implantable lead of example 21, wherein the one or more polymers comprise a first polymer and a second polymer, and wherein the first polymer defines a different durometer hardness than the second polymer.

### Example 23

The implantable lead of example 22, wherein the first polymer is disposed radially inwards of the second polymer, and wherein the first polymer defines a smaller durometer hardness than the second polymer.

### Example 24

The implantable lead of any of examples 22 and 23, wherein the first polymer defines a first durometer, wherein the second polymer defines a second durometer, and wherein one or more of the first durometer and the second durometer is about 50 durometers to 75 durometers.

### Example 25

The implantable lead of any of examples 17-24, wherein each flexible region comprises a plurality of third regions, each third region being connected to one first region of a plurality of first regions and to one second region of a plurality of second regions.

### Example 26

The implantable lead of claim any of examples 17-24, wherein the third portion defines the third outer diameter at a longitudinal center of the second portion.

### Example 27

The implantable lead of example 26, wherein, for each flexible region, the longitudinal center of the second portion is disposed at a longitudinal center of the respective flexible region.

### Example 28

The implantable lead of any of examples 17-27, wherein one or more of the first outer diameter or the third outer diameter is between 1.02 millimeters (mm) and 1.27 mm.

### Example 29

The implantable lead of any of examples 17-28, wherein the second outer diameter is between 0.91 mm and 1.02 mm.

### Example 30

A method of manufacturing an implantable lead configured to be placed near a nerve of a patient, comprising: forming an electrically conductive material into a plurality of electrodes, each electrode of the plurality of electrodes defining an elongated body configured to extend along a longitudinal axis of the lead; and forming an elongated shaft of the implantable lead along a longitudinal axis, the elongated shaft comprising: the plurality of electrodes; and a plurality of flexible regions connecting the plurality of electrodes, each flexible region of the plurality of flexible regions being configured to be disposed between longitudinally adjacent electrodes of the plurality of electrodes, and wherein each flexible region comprises: a distal portion defining a first diameter; a proximal portion defining a second diameter; and a medial portion connecting the distal portion and the proximal portion and defining a third diameter at a narrowest location along a longitudinal length of the flexible region, wherein each flexible region defines a concave cylindrical shape along the longitudinal length of the flexible region.

### Example 31

The method of example 30, wherein each flexible region is configured to be connected to a first electrode of the plurality of electrodes at a distal end and to a second electrode of the plurality of electrodes at a proximal end.

### Example 32

The method of any of examples 30 and 31, wherein, for each flexible region, the concave cylindrical shape is symmetrical along the longitudinal length of the flexible region.

### Example 33

The method of any of examples 30-32, wherein forming each flexible region comprises: disposing a first polymer over the conductor coil, and between the longitudinally adjacent electrodes, wherein the first polymer is connected to an end of each electrode of the longitudinally adjacent electrodes; and disposing a second polymer over the first polymer, wherein the second polymer is connected to the end of each electrode of the longitudinally adjacent electrodes.

### Example 34

The method of example 33, wherein the first polymer defines a different durometer hardness than the second polymer.

### Example 35

The method of example 34, wherein the first polymer defines a smaller durometer hardness than the second durometer.

### Example 36

The method of any of examples 34 and 35, wherein the durometer hardness of one or more of the first polymer or the second polymer is between 50 durometers and 75 durometers.

### Example 37

The method of any of examples 30-36, wherein the first diameter is equal to the second diameter.

### Example 38

The method of any of examples 30-37, wherein one or more of the first diameter or the second diameter is between 1.02 millimeters (mm) to 1.27 mm.

### Example 39

The method of any of examples 30-38, wherein the third diameter is between 0.91 mm to 1.02 mm.

### Example 40

The method of any of examples 30-39, wherein a first length of the distal portion along the longitudinal axis is greater than a second length of the proximal portion along the longitudinal axis.

### Example 41

The method of any of examples 30-40, wherein a first length of the distal portion along the longitudinal axis is less than a second length of the proximal portion along the longitudinal axis.

### Example 42

The method of any of examples 30-41, wherein for each flexible region, the concave cylindrical shape defines a continuous curvature along the longitudinal axis.

### Example 43

The method of any of examples 30-41, wherein for each flexible region, the concave cylindrical shape defines a stepwise change in an outer diameter of the flexible region along the longitudinal axis.

### Example 44

The method of any of examples 30-43, wherein for each flexible region, the concave cylindrical shape reduces strain at a distal end and a proximal end of the flexible region in response to application of a bending force on the lead along a plane normal to the longitudinal axis.

Various examples have been described. These and other examples are within the scope of the following claims.

1-15. (canceled)

16. An implantable lead configured to be placed near a nerve of a patient, the implantable lead comprising:
an elongated shaft defining a longitudinal axis and an inner lumen, wherein the elongated shaft comprises:
a plurality of electrodes disposed on the elongated shaft; and
a plurality of flexible regions, each flexible region defining a concave cylindrical shape along the longitudinal axis, wherein each flexible region separates longitudinally adjacent electrodes of the plurality of electrodes, and wherein the concave cylindrical shape comprises:
a distal portion defining a first diameter;
a proximal portion defining a second diameter; and
a concave portion connecting the distal portion and the proximal portion and defining a third diameter at a narrowest location along the concave portion, wherein the third diameter is less than the first diameter or the second diameter; and
a conductor coil disposed within the inner lumen and extending along the longitudinal axis, wherein the conductor coil comprises a plurality of conductive wires, and wherein each electrode of the plurality of electrodes is electrically connected to a respective wire of the plurality of conductive wires.

17. The implantable lead of claim 16, wherein each flexible region comprises a first polymer and a second polymer, wherein the first polymer defines a different durometer hardness than the second polymer.

18. The implantable lead of claim 17, wherein the first polymer is disposed radially inwards of the second polymer, and wherein the first polymer defines a smaller durometer hardness than the second polymer.

19. The implantable lead of claim 17, wherein a first durometer of the first polymer is greater than a second durometer of the second polymer.

20. The implantable lead of claim 17, wherein the first polymer defines a first durometer, wherein the second polymer defines a second durometer, and wherein one or more of the first durometer and the second durometer is between 50 durometers and 75 durometers.

21. The implantable lead of claim 16, wherein one or more of the first diameter or the second diameter is about 1.02 millimeters (mm) to about 1.27 mm.

22. The implantable lead of claim 16, wherein the third diameter is about 0.91 mm to about 1.02 mm.

23. The implantable lead of claim 16, wherein a first length of the distal portion along the longitudinal axis is greater than a second length of the proximal portion along the longitudinal axis.

24. The implantable lead of claim 16, wherein a first length of the distal portion along the longitudinal axis is less than a second length of the proximal portion along the longitudinal axis.

25. The implantable lead of claim 16, wherein one or more flexible regions of the plurality of flexible regions is symmetrical along the longitudinal axis.

26. The implantable lead of claim 16, wherein, for each flexible region, the concave cylindrical shape defines a continuous curvature along the longitudinal axis.

27. The implantable lead of claim 16, wherein, for each flexible region, the concave cylindrical shape defines a stepwise change in an outer diameter of the flexible region along the longitudinal axis.

28. The implantable lead of claim 16, wherein each electrode of the plurality of electrodes defines an elongated annulus extending along the longitudinal axis, wherein each electrode defines an opening disposed on the elongated annulus, and wherein each electrode is electrically connected to the conductor coil via a conductive wire secured within the opening.

29. The implantable lead of claim 16, wherein, for each flexible region, the concave cylindrical shape increases strain at a concave portion and reduces strain at a distal end and a proximal end of the flexible region in response to application of a bending force on the lead along a plane normal to the longitudinal axis.

30. An implantable lead configured to be placed near a nerve of a patient, the implantable lead comprising:
an elongated shaft defining a longitudinal axis and comprising:
a plurality of electrodes; and
a plurality of separate flexible regions arranged along the longitudinal axis, each flexible region extending along the longitudinal axis from a proximal end to a distal end and comprising:
a first portion defining a first outer diameter;
a second portion defining a second outer diameter;
a third portion connecting the first portion to the second portion and defining a third outer diameter,
wherein the third outer diameter is less than the first outer diameter or the second outer diameter.

31. The implantable lead of claim 30, wherein each flexible region defines a first step between the first portion and the third portion and a second step between the second portion and the third portion, wherein the first step defines a first change in an outer diameter of the respective flexible region, and wherein the second step defines a second change in the outer diameter of the respective flexible region.

32. The implantable lead of claim 30, wherein each flexible region comprises a first polymer and a second polymer, wherein the first polymer is disposed radially inwards of the second polymer, and wherein the first polymer defines a smaller durometer hardness than the second polymer.

33. The implantable lead of claim 32, wherein the first polymer defines a first durometer, wherein the second polymer defines a second durometer, and wherein one or more of the first durometer and the second durometer is about 50 durometers to 75 durometers.

34. The implantable lead of claim 30, wherein each flexible region comprises a plurality of third regions, each third region being connected to one first region of a plurality of first regions and to one second region of a plurality of second regions.

35. The implantable lead of claim 30, wherein the third portion defines the third outer diameter at a longitudinal center of the second portion.
